SEQUENCE LISTING (26 sequences)

SEQ ID NO: 1 - protein, 24 aa, Artificial Sequence
  Synthetic
TTEXESXNXGYXFGQEEETYNIVA

SEQ ID NO: 2 - protein, 24 aa, Artificial Sequence
  Synthetic
TTEXESXNXGYEFGQEEETYNIVA

SEQ ID NO: 3 - protein, 24 aa, Synechocystis PCC6803
TTEVESQNYGYKFGQEEETYNIVA

SEQ ID NO: 4 - protein, 24 aa, Synechocystis PCC6803
TTEVESQNYGYKFGQEEETYNIVA

SEQ ID NO: 5 - protein, 24 aa, Synechocystis PCC6803
TTEVESQNQGYKFGQEEETYNIVA

SEQ ID NO: 6 - protein, 24 aa, Nicotiana tabacum
TTENESANEGYRFGQEEETYNIVA

SEQ ID NO: 7 - protein, 24 aa, Solanum lycopersicum
TTENESANEGYRFGQEEETYNIVA

SEQ ID NO: 8 - protein, 24 aa, Spinacia oleracea
TTENESANEGYRFGQEEETYNIVA

SEQ ID NO: 9 - protein, 24 aa, Arabidopsis thaliana
TTENESANEGYRFGQEEETYNIVA

SEQ ID NO: 10 - protein, 24 aa, Zea mays
TTENESANEGYKFGQEEETYNIVA

SEQ ID NO: 11 - protein, 24 aa, Oryza sativa
TTENESANEGYRFGQEEETYNIVA

SEQ ID NO: 12 - protein, 24 aa, Triticum aestivum
TTENESANEGYRFGQEEETYNIVA

SEQ ID NO: 13 - protein, 24 aa, Hordeum vulgare
TTENESANEGYKFGQEEETYNIVA

SEQ ID NO: 14 - protein, 24 aa, Physcomitrella patens
TTENESANAGYKFGQEEETYNIVA

SEQ ID NO: 15 - protein, 24 aa, Adiantum capillus-veneris
TTENESANAGYKFGQEEETYNIVA

SEQ ID NO: 16 - protein, 24 aa, Chlamydomonas reinhardtii
TTENESANEGYRFGQEEETYNIVA

SEQ ID NO: 17 - protein, 24 aa, Thermosynechococcus elongatus
TTETESANYGYKFGQEEETYNIVA

SEQ ID NO: 18 - protein, 24 aa, Thermosynechococcus elongatus
TTETESANYGYKFGQEEETYNIVA

SEQ ID NO: 19 - protein, 24 aa, Thermosynechococcus elongatus
TTETESTNYGYKFGQEEETYNIVA

SEQ ID NO: 20 - protein, 24 aa, Prochlorococcus cyanophage
TTEQESQNYGYRFGQEEETYNIVA

SEQ ID NO: 21 - DNA, 1795 bp, Artificial Sequence
  Synthetic
gcttggctggactgacactaaaactcggtgacttttcatcgtggaaaggacataggccaaggtgatcccgcccccttttcttgagcacgacataatcgccgataatttcgacaatgtctattctttgcttaatttcttggatggtgtctggatgcagacggaggttatccatgataaatcaacctactctccttctaagagtgacgatggggagagatacttgtcaacttaagctctactcaagcatggtcaaccagcaaaaagcaccccccggtgaaggagagtgcaatttgcagattattcagttggcattacaccaaggaattaaccgttgacagcaggagcggtcaaagccacaggagcttgctccccagacgctaagtcgagggggaagttgtgggcattgcgttcgtgcattacttcaaaaccgatgttggcgcggttcaatacatcagcccaggtgccgattacacggccttggctatccaagatggactggttgaagttgaaaccgttcaggttgaacgccatggtgcttacacccatagcagtgaaccagatgccgattacaggccaagcacccaagaagaagtgcaaggaacggctgttgttgaaagaagcatattggaagatcaaccgaccaaagtagccgtgggcggcaacgatgttgtaggtttcttcttcttgaccgaatttgtaaccgtagttctgggattcaacttcggtggtttcacgcaccaaggaggaggttaccaaggaaccgtgcatggcggagaacaagctaccaccgaatacaccagccacacctaacatgtggaaggggtgcatcaggatgttgtgctcagcttggaacacgatcatgaagttgaaggtaccagaaatacccaagggcataccatcagagaaggagccttgaccaatggggtagatcaagaatacagcggtggcagcggatacgggggcagagtaagccacacaaatccaaggacgcatacctaagcggtaggaaagttcccactgacgacccatgtagcagaaaatgccgatgaggaagtggaatactaccaactggtaaggaccaccgttgtacaaccactcatctaaggaagcggcttcccagatggggtagaagtgcaaaccgatagcgttggaagaaggtacaacagcaccagagatgatgttgttaccgtaaagcaaagaaccagcaacgggctcacggataccgtcgatgtcaacggggggagcggcgatgaaggcaatgatgaagcaagtggtggcagttaagagggtggggatcatcaaggtaccgaaccaaccgacataaatccggttgttggtagaggtcacccactgacaaaactgttcccacaatgaagcgctttcgcgctgttggagagtcgttgtcatttggttataattccttatgtatttgtcaatgttcaaaggatttggcctcaagctcagagatttctttgcttgggagccggttatgtatatgagtaatataacaagattgtaacaatttgtaaatagcttgctgaaaaaaatctgtctagccggtggttgctaggggttgccgatcgccattagaggggtgattgattccgttggggcttcagtctgtggtggtggggagatggaaagccactaatcctgcaacacaacttccccttggcgcagaatttggaagatttcctttgctccgtcccattgggcaacggtggacggtagacctttgccaattggcccctgttgctccaggctgaggcaatctagacaatgaacggcagggaaa

SEQ ID NO: 22 - DNA, 20 bp, Artificial Sequence
  Synthetic
gcttggctggactgacacta

SEQ ID NO: 23 - DNA, 18 bp, Artificial Sequence
  Synthetic
tttccctgccgttcattg

SEQ ID NO: 24 - DNA, 41 bp, Artificial Sequence
  Synthetic
cccagaactacggttacgcattcggtcaagaagaagaaacc

SEQ ID NO: 25 - DNA, 41 bp, Artificial Sequence
  Synthetic
cccagaactacggttacgaattcggtcaagaagaagaaacc

SEQ ID NO: 26 - DNA, 41 bp, Artificial Sequence
  Synthetic
cccagaactacggttacctcttcggtcaagaagaagaaacc

(19) United States
(12) Patent Application Publication
(10) Pub. No.: US 2014/0223610 A1
(43) Pub. Date: Aug. 7, 2014

(54) BIO-ENGINEERED PHOTOSYSTEMS

(71) Applicant: TECHNION RESEARCH AND DEVELOPMENT FOUNDATION, LTD., HAIFA (IL)

(72) Inventors: Noam ADIR, Kiryat Tivon (IL); Gadi SCHUSTER, Kiryat Tivon (IL); Shirley LAROM, Zichron Ya'akov (IL); Faris SALAMA, Qalansawa (IL)

(73) Assignee: TECHNION RESEARCH AND DEVELOPMENT FOUNDATION, LTD., HAIFA (IL)

(21) Appl. No.: 14/256,262

(22) Filed: Apr. 18, 2014

Related U.S. Application Data
(62) Division of application No. 12/954,002, filed on Nov. 24, 2010.
(60) Provisional application No. 61/263,828, filed on Nov. 24, 2009.

(51) Int. Cl. C12N 15/82; C12N 15/79

(52) U.S. Cl. CPC C12N 15/8261; C12N 15/79
USPC 800/306; 800/298; 435/257.2; 800/317.3; 800/317.4; 800/320.1; 800/320.2; 800/320.3; 800/320; 800/317.2

(57) ABSTRACT

The present invention relates to bio-engineered photosystems, specifically photosystem II (PSII) having an alternative electron transfer pathway that enables electron flow from PSII to a water-soluble protein electron carrier. The present invention further relates to methods and systems for electron transfer using the bio-engineered photosystems. Such photosystems may be utilized for electrical energy production, hydrogen production and/or reduction of carbon-based gases (for example, CO₂ and CO) to liquid fuels.

### CROSS-REFERENCE TO RELATED APPLICATIONS

This application is a divisional of application Ser. No. 12/954,002, filed Nov. 24, 2010, which claims the benefit of Provisional Patent Application No. 61/263,828 filed Nov. 24, 2009, the contents of which are incorporated by reference herein in its entirety.

### FIELD OF THE INVENTION

The present invention relates to bio-engineered photosystems, specifically photosystem II (PSII) having an alternative electron transfer pathway that enables electron flow from PSII to a water-soluble protein electron carrier. The present invention further relates to methods and systems for electron transfer using the bio-engineered photosystems. Such photosystems and methods may be utilized for electrical energy production, hydrogen production and/or reduction of carbon-based gases (for example, CO₂ and CO) to liquid fuels.

### BACKGROUND OF THE INVENTION

In an age of ever progressive depletion of resources and environmental concerns, alternative energy sources are continuously sought. One approach is to develop means to harness the natural process of energy production used in photosynthetic organisms.

Photosynthesis is the major source of useful chemical energy in the biosphere, occurring in plants, algae, and many species of bacteria. The photosynthetic process includes two stages. In the first stage, light-dependent electron transfer reactions are utilized for proton gradient formation, which in turn is used for the production of ATP and other reducing equivalents, such as NADPH. In the second stage, light-independent reactions use the products of the first stage to capture and reduce carbon dioxide.

During the first stage, light energy is absorbed by light harvesting antenna complexes containing pigment molecules. In plants, light is absorbed primarily using the pigment chlorophyll. Light energy, absorbed by the light harvesting antenna complexes, is transferred to photochemical reaction centers (RC), initiating charge separation on specific chlorophyll molecules bound to the RC proteins. Following charge separation, electrons are transferred sequentially to multiple acceptor molecules, each with a redox potential determined by its immediate environment. One common feature of all electron transfer pathways is the requirement for insulation of the redox active cofactors from potentially reducing/oxidizing molecules within the RC or in the surrounding media. Insulation provides the system with maximal electron transfer rates and efficiencies, and also prevents damage to the RC.

The source of electron replenishment in a photosynthesis system differs according to the reaction center type. In purple non-sulfur bacteria, for example, electrons are cycled back to the reaction center by water-soluble electron carriers, for example, a cytochrome c type protein. In oxygenic photosynthetic organisms, including cyanobacteria, red and green algae and plants, electron flow is non-cyclic, and occurs in two steps that involve two photosystems: Photosystem I (PSI) and Photosystem II (PSII). In this type of reactions, the deficit of electrons is replenished by electrons taken from water molecules.

The initial steps of oxygenic photosynthetic electron transfer occur within PSII, which is a complex composed of proteins, pigments and cofactors, located within the thylakoid membranes. PSII has a redox potential of between 1.2V to 1.8V, required to extract electrons from water molecules. The site of water oxidation and other components of PSII are illustrated in FIG. 1A. The process of electron transfer in PSII includes the following steps:

Upon illumination, a P₆₈₀ chlorophyll is photoexcited. The photoexcited P₆₈₀ transfers electrons via intermediate cofactors called pheophytin a and plastoquinone A (PQ, Q_{A}) in order to finally doubly reduce a transiently bound PQ molecule (Q_{B}). Q_{B}²⁻ is protonated and released from the RC into the thylakoid membrane. The redox active cofactors that enable electron transfer from water to the secondary quinone acceptor Q_{B}, are mainly embedded within two proteins called D1 and D2. Under normal conditions of illumination, the D1 protein of the RC core is irreversibly damaged over time and is replaced in a fashion that preserves the integrity of the PSII complex.

Development of methods and systems that utilize the principles of the photosynthetic process for energy production is a major scientific and technological challenge. For example, synthetic and semi-synthetic systems based on photosynthetic processes have been proposed. These include various attempts to use dyes bound to solid-state materials and coupled molecules that form novel electron transfer pathways. Other examples of technologies for solar energy conversion include semiconductor/liquid junction solar cells and photovoltaic cells. Systems based on natural biological material have also been proposed. However, one of the major limitations of direct use of photosynthetic organisms or their components for solar energy conversion is that natural biological material has a relatively short functional life time. The use of biological material for energy production is currently limited to the production of bio-fuels—photosynthetic organisms, such as plants, green algae and cyanobacteria, are grown and then converted to fuel materials, for example, ethanol.

There still remains a need for cost effective, non-polluting systems and methods to convert sunlight to electrical energy. For example, it would be highly beneficial to have stable and efficient bio-engineered photosystems for generating electrical current, for hydrogen production and/or for reduction of carbon-based gases to liquid fuels.

### SUMMARY OF THE INVENTION

The present invention provides bio-engineered photosystem II complexes comprising a variant D1 protein, which are capable of transferring electrons to water-soluble electron carrier proteins. The present invention further provides isolated thylakoid membranes and genetically modified photosynthetic organisms comprising the variant D1 proteins, and methods and systems for electron transfer utilizing same.

The present invention discloses for the first time a specific mutation in the D1 protein of PSII that results in the formation of a novel electron conduit, which enables electrons to flow from PSII to water-soluble electron carrier proteins. During the natural process of photosynthesis, PSII extracts electrons from water and moves them through the thylakoid membrane that isolates the electricity flow and keeps it from escaping. It is now disclosed that a single modification to the D1 protein enables electrons to flow out of the membrane instead of being trapped inside it. Such a modification has been exemplified herein by site directed mutagenesis of the D1 protein.

The electrons extracted by PSII comprising the variant D1 of the present invention may be utilized for electrical energy production. In addition, protons resulting from the oxidation of water molecules may be utilized for the production of molecular hydrogen or other fuels. Thus, photosystems and methods of the present invention may be utilized, for example, for electrical energy production, hydrogen production and/or reduction of carbon-based gases (for example, CO₂ and CO) to liquid fuels.

Typically, a D1 protein comprises the following amino acid consensus sequence:

| | |
|---|---|
| TTEX₁ESX₂NX₃GYX₄FGQEEETYNIVA | (SEQ ID NO.: 1) |

wherein:

X₁ represents valine, asparagine, threonine or glutamine;

X₂ represents glutamine, alanine or threonine;

X₃ represents tyrosine, glutamine, glutamate or alanine; and

X₄ represents lysine or arginine.

The present invention now discloses variants of the D1 protein having an amino acid other than a positively-charged amino acid at the position indicated as X₄. Preferably, the original amino acid present at position X₄ of the consensus sequence is substituted with an acidic amino acid, for example glutamate or aspartate. According to other embodiments the naturally occurring basic amino acid may be replaced with any other acidic amino acid or any equivalent synthetic analogue. In some typical embodiments, the original amino acid present at position X₄ of the consensus sequence is substituted with a glutamate residue. Without wishing to be bound by any particular theory or mechanism, a substitution of a basic amino acid by an acidic residue at position X₄, which is located on the cytoplasmic surface of D1, creates a binding site for soluble redox active proteins, for example, cytochrome c. The engineered site is located at or near the vicinity of the Q_{A} intermediate acceptor site within PSII. The close proximity between the Q_{A} and the bound electron carrier protein may allow electrons to flow from a reduced Q_{A} to the soluble redox active proteins.

Even though the engineered site is highly conserved among all oxygenic photosynthetic organisms, and therefore its modification might be deleterious to the protein function, it was surprisingly found that the new pathway does not alter the ability of PSII to perform natural photosynthetic electron transfer, and the resulting organism maintains photoautotrophic growth. In addition, the variant D1 was found to have increased stability against photodamage in the presence of a water-soluble protein electron carrier.

According to one aspect, the present invention provides an isolated variant D1 protein comprising a mutated SEQ ID NO.: 1 consensus sequence, wherein the amino acid present at the position indicated as X₄ in the consensus sequence set forth in SEQ ID NO.: 1 is substituted with glutamate.

Thus, the resulting D1 variant comprises the following amino acid sequence:

| | |
|---|---|
| TTEX₁ESX₂NX₃GYEFGQEEETYNIVA. | (SEQ ID NO.: 2) |

wherein:

X₁ represents valine, asparagine, threonine or glutamine;

X₂ represents glutamine, alanine or threonine; and

X₃ represents tyrosine, glutamine, glutamate or alanine.

In some embodiments, the consensus sequence corresponds to positions 227-250 of the D1 protein. According to these embodiments, the variant D1 protein comprises a substitution to glutamate at position 238 of D1.

In other embodiments, the consensus sequence corresponds to positions 197-220 of the D1 protein. According to these embodiments, the variant D1 protein comprises a substitution to glutamate at position 208.

In some embodiments, the original, naturally-occurring, amino acid present at the position indicated as X₄ in the consensus sequence set forth in SEQ ID NO.: 1 is lysine. According to these embodiments, an isolated variant D1 protein is provided, the variant comprises a lysine to glutamate substitution at the position indicated as X₄ in the consensus sequence set forth in SEQ ID NO.:1.

In other embodiments, the original, naturally-occurring, amino acid present at the position indicated as X₄ in the consensus sequence set forth in SEQ ID NO.: 1 is arginine. According to these embodiments, an isolated variant D1 protein is provided, the variant comprises an arginine to glutamate substitution at the position indicated as X₄ in the consensus sequence set forth in SEQ ID NO. 1.

D1 proteins of all oxygenic photosynthetic organisms may be modified to include the amino acid substitutions disclosed herein. Oxygenic photosynthetic organisms include all photosynthetic organisms that use water as an electron source, and generate oxygen as an end product of photosynthesis, for example, plants, algae and cyanobacteria.

In some embodiments, D1 proteins of plants are modified according to embodiments of the present invention. D1 proteins of both dicotyledons and monocotyledons can be modified according to embodiments of the present invention. In some embodiments, the plants include species of tobacco, tomato, spinach, Arabidopsis, maize, rice, wheat, barley, potato, carrot, cabbage, Physcomitrella and Adiantum. Each possibility represents a separate embodiment of the invention.

In other embodiments, D1 proteins of algae are modified according to embodiments of the present invention. In some typical embodiments, the algae belong to the Chlorophyta phylum (chlorophytes). In some embodiments, the algae include species of Chlamydomonas, Chlorella and Spirogyra. Each possibility represents a separate embodiment of the invention.

In yet other embodiments, D1 proteins of cyanobacteria are modified according to embodiments of the present invention. In some embodiments, the cyanobacteria include species of Synechocystis, for example Synechocystis sp. PCC 6803, Thermosynechococcus, for example T. elongatus or T. vulcanus and Prochlorococcus. Each possibility represents a separate embodiment of the invention.

According to another aspect, the present invention provides an isolated PSII comprising a variant D1 protein, the variant comprises a substitution to glutamate at the position indicated as X₄ in the consensus sequence set forth in SEQ ID NO.: 1.

PSII complexes of the present invention enable electron transfer to a water-soluble protein electron carrier.

In some embodiments, the water-soluble protein electron carriers include cytochromes, ferredoxins (including rubredoxins), plastocyanins and flavocytochromes. Each possibility represents a separate embodiment of the invention.

In some exemplary embodiments, the cytochromes include cyt c, cyt c₂ and cyt c₆. Each possibility represents a separate embodiment of the invention.

In some typical embodiments, cytochrome c is used as the soluble protein electron carrier.

According to yet another aspect, the present invention provides an isolated thylakoid membrane comprising at least one PSII having the variant D1 of the present invention.

Thylakoid membranes may be obtained from any oxygenic photosynthetic organism, as detailed above. As exemplified herein below, isolated thylakoid membranes of the present invention are able to perform light-dependent reduction of protein electron carrier, for example, cytochrome c, with water as the electron donor.

The present invention further encompasses genetically modified oxygenic photosynthetic organisms comprising the variant D1 of the present invention. The gene encoding the wild-type D1 has been mutated in these organisms, such that the mutated gene encodes a variant D1 comprising a substitution to glutamate at the position indicated as X₄ in the consensus sequence set forth in SEQ ID NO.: 1.

Thus, according to yet another aspect, the present invention provides a genetically modified oxygenic photosynthetic organism comprising a mutated D1 gene, the mutated D1 gene encodes a variant D1 comprising a substitution to glutamate at the position indicated as X₄ in the consensus sequence set forth in SEQ ID NO.: 1.

In some embodiments, more than one copy of the gene encoding the wild-type D1 is present in the genome of the oxygenic photosynthetic organism or its chloroplast genome. According to these embodiments, a genetically-modified oxygenic photosynthetic organism is provided, wherein at least one copy of the gene encoding the wild-type D1 protein has been mutated. The mutated gene encodes a variant D1 that comprises a substitution to glutamate at the position indicated as X₄ in the consensus sequence set forth in SEQ ID NO.: 1.

Any oxygenic photosynthetic organism may be used according to embodiments of the present invention, as detailed above.

In some embodiments, the genetically modified oxygenic photosynthetic organism is a unicellular organism. In other embodiments, the genetically modified oxygenic photosynthetic organism is a multicellular organism. According to these embodiments, a genetically modified oxygenic photosynthetic organism is provided, the organism comprises at least one cell comprising a mutated D1 gene, wherein the mutated D1 gene encodes a variant D1 comprising a substitution to glutamate at the position indicated as X₄ in the consensus sequence set forth in SEQ ID NO.: 1. It is to be understood that “at least one cell” refers to a cell capable of performing photosynthesis. For example, if the organism is a plant, cells capable of performing photosynthesis include, but are not limited to, cells of leaves and stems.

In some embodiments, the genetically modified oxygenic photosynthetic organism is a cyanobacterium. In other embodiments, the genetically modified oxygenic photosynthetic organism is an alga. In yet other embodiments, the genetically modified oxygenic photosynthetic organism is a plant.

According to yet another aspect, the present invention provides a method for electron transfer comprising combining:
i) a PSII having a variant D1 protein, the variant comprises a substitution to glutamate at the position indicated as X₄ in the consensus sequence set forth in SEQ ID NO.: 1; and
ii) a water-soluble protein electron carrier,
   thereby generating an electron transfer chain enabling electrons to flow from PSII to the water-soluble protein electron carrier.

In some embodiments, the method further comprises combining an inhibitor of the Q_{B} site within D1. In some embodiments, the inhibitor of the Q_{B} site is selected from the group consisting of DCMU, atrazine-type inhibitor, bromoxynil-type inhibitor and substituted 9,10-anthraquinone.

The atrazine-type inhibitors include, but are not limited to, triazines. The bromoxynil-type inhibitors include, but are not limited to, nitriles.

According to a further aspect, the present invention provides a system for electron transfer comprising a PSII comprising the variant D1 protein of the present invention.

According to a further aspect, the present invention provides a system for electron transfer comprising a MI comprising the variant D1 protein of the present invention and a water-soluble protein electron carrier.

In some embodiments, the system further comprises an inhibitor of the Q_{B} site within D1.

These and further aspects and features of the present invention will become apparent from the figures, detailed description, examples and claims which follow.

### BRIEF DESCRIPTION OF THE FIGURES

FIG. 1. In silico analysis of the cytoplasmic-facing surface of PSII. A. Schematic model of PSII reaction center. Gray and black boxes represent D2 and D1 proteins, respectively, electron transfer chain co-factors are depicted in stick form. White arrows indicate the direction of electron flow from the oxygen evolving center (depicted as “4Mn/Ca”) to Q_{B}. Gray arrow and gray curved surface indicate a potential position for an engineered electron transfer pathway. The D1 and D2 proteins crossover each other on the cytoplasmic surface, and the position of the conserved positive residue is indicated (D1-K238). Y_{z}— tyrosine-Z; Pheo—pheophytin; Q_{A}—plastoquinone A; Q_{B}—plastoquinone B. B. Electrostatic potential mapped onto the cytoplasmic face of wild type PSII using the 3BZ1 crystal structure. Light and dark gray indicate negative and positive potentials, respectively. The PSII reaction center is identified by the white oval, and the D1-K238 site by the black oval C. Close-up of the D1-K238 potential (black circle) positioned above the Q_{A} site D. Calculated electrostatic potential mapped onto the stromal surface of the Glu virtual mutant. A negative potential appears at the position of D1-Glu238 (black circle).

FIG. 2. Alignment of amino acid sequences of D1 proteins from various oxygenic photosynthetic organisms. The shown sequences correspond to amino acids nos. 227 to 250 of the D1 protein, except for Prochlorococcus cyanophage, in which the shown sequence corresponds to amino acids nos. 197-221 of the D1 protein. Bracketed residue indicates the position of the conserved positive residue. The alignment was performed using the ClustalW2 tool. The organisms are as follows: “Synechocystis psbA1-3” are the three psbA genes found in Synechocytis PCC sp. 6803 with the accession numbers NP_{—}443017.1, NP_{—}439906.1 and NP_{—}441550.1, respectively; Tobacco, Nicotiana tabacum NP_{—}054477.1; Tomato, Solanum lycopersicum YP_{—}514832.1; Spinach, Spinacia oleracea NP_{—}054912.1; Arabidopsis, Arabidopsis thaliana NP_{—}051039.1; Maize, Zea mays NP_{—}043004.1; Rice, Oryza sativa Japonica NP_{—}039360.1; Wheat, Triticum aestivum NP_{—}114239.1; Barley, Hordeum vulgare YP_{—}874633; Physcomitrella, Physcomitrella patens NP_{—}904209.1; Adiantum, Adiantum capillus-veneris NP_{—}848104.1; Chlamydomonas, Chlamydomonas reinhardtii NP_{—}958413.1; “T. elongatus psbA1-3” are the three psbA genes found in Thermosynechococcus elongatus NP_{—}682633.1, NP_{—}682633.1 and NP_{—}682267.1, respectively; Prochlorcoccus cyanopage, Prochlorococcus cyanophage ABF55978.

FIG. 3. Construction of the psbA3 mutant strains. A. A schematic presentation of the Synechocystis PCC 6803 genomic region containing the RSS psbA3 fragment. The mutations locus is marked with a vertical arrow. A partial nucleotide sequence of the psbA3 gene is presented with the corresponding amino acid at position 238. Recognition sequences of restriction enzymes are underlined and the enzymes are detailed above the sequence. B. PCR analysis to verify the location of each mutation in the genome of the mutant strain. The amplification was performed using the primers shown in Table 1. The 1856 bp PCR product, amplified from the genomic DNA of each strain, was digested with the different restriction enzymes as detailed below each lane. Each strain was validated in addition by DNA sequencing. C. Selection of Synechocystis TD34 strain transformed with the psbA3 gene (shown here for the RSS strain) that replaces the spectinomycin cassette as shown in the schematic representation of the TD34 genome shown on the right. Growth without glucose indicates that the resulting strain is photosynthetic.

FIG. 4. Photosynthetic growth and protein complex analysis of mutant strains. A. Selection for photosynthetic growth of mutant strains. After transformation, cells were grown on BG-11 media supplemented with antibiotics (kanamycin and chloramphenicol). Mutant strains are indicated below each well. B. Immunoblot of isolated thylakoid membranes (1 g chlorophyll/lane). Strains are indicated on the top of each lane. The antibodies probed for targets representing the major photosynthetic complexes in the thylakoid membrane (indicated on left). The TD34 strain, lacking all three psbA genes is also deficient in the amount of PSI.

FIG. 5. Photosynthetic activities of Synechocystis strains. Oxygen evolution rates in whole cells (gray) and photoreduction of DCIP by isolated membranes (black) were measured as described in the text. RSS contains the wt psbA3. Ala, Leu and Glu are three site directed mutations at residue 238 of psbA3 gene of Synechocystis. Values are the average of three independent measurements.

FIG. 6. A. Similar degradation rate of D1 in the RSS and the mutated strains. Autoradiograms of thylakoid membranes of Synechocystis strains (WT, RSS and Ala shown in the figure) following a pulse-labeling in the presence of 5-methionine and a chase for 60, 90 and 120 min in the presence of non-radioactive methionine, both under 850 μE/(m²*sec)). The D1 in wild type cells, containing the three psbA genes, is degraded slower than the RSS (containing only the psbA3 gene) and the mutated strains (containing modified psbA3). However, no differences were observed between the RSS and the mutated strains Immunoblot of the samples are shown below the autoradiograms, displaying similar accumulation of D1. B. Quantification of the radioactive D1 bands. The quantification was performed by quantitative analysis of the D1 signal in autoradiographs, such as the one shown in panel A. It is shown that there are no differences between the RSS and the mutated strains.

FIG. 7. Photoreduction of cytochrome c. A. Thylakoid membranes from the RSS strain or Glu mutant strain were illuminated in the presence of oxidized cytochrome c for 4 minutes, with or without the addition of DCMU, and the light-dark difference absorption spectrum was measured. cyt cₒₓ, spectra of oxidized cyt c, is shown for reference. B. Quantification of cyt c photoreduction by RSS (white bars) or Glu (black bars) membranes in three independent experiments in the absence or presence of DCMU.

FIG. 8. Light-dependent electron transfer from H₂O to cyt c in the Glu strain. Oxygen evolution by isolated thylakoids from the RSS and Glu strains was measured. For samples 1 and 2, DCPIP served as the exogenous electron acceptor without (1) or with (2) the addition of DCMU. In sample 3, cytochrome c (in the presence of DCMU) was the exogenous acceptor

FIG. 9. Evaluation of D1 photodamage-induced crosslinking in isolated thylakoids illuminated for 0, 30, 60, or 90 minutes under light intensity of 850 μE/(m₂s). A. RSS strain. The left panel shows immunoblot of the D1 protein in thylakoids isolated from the RSS strain. The membranes were illuminated either in the absence or the presence of oxidized (oxy) cytochrome c. The right panel shows quantification of the rates of D1 disappearance in the RSS strains in the absence or presence of oxidized cytochrome c (cyt). B. Glu mutant strain. The top left panel shows immunoblot of the D1 protein in thylakoids isolated from the Glu mutant strain. The membranes were illuminated either in the absence or the presence of oxidized (oxy) or reduced (red) cytochrome c. The bottom left panel shows analysis of thylakoids purified from the Glu mutant and illuminated as described above, by spotting on nitrocellulose membrane that was decorated with antibodies to the D1. In this method, the total amount of D1, including the non-crosslinked and the crosslinked species, is determined. Thus, it is possible to observe that the disappearance of the D1 from the 32 kDa region of the gel in illuminated thylakoids is not because of its degradation but rather by its crosslinking. The right panel shows quantification of the rates of D1 disappearance in the Glu mutant strain in the absence or presence of oxidized or reduced cytochrome c (cyt). Quantification of the D1 signal from the dot blot following 90 min of illumination described above is also shown. FIG. 10. D1 aggregation upon illumination of the thylakoids. Thylakoids purified from Glu mutant cells were photoinhibited as described in FIG. 9B. At the time indicated, thylakoids were fractionated by SDS-PAGE and the D1 was detected by immunobloting. An over exposure of the D1 signal, in order to observe the crosslinked aggregates, is presented. The identity of the crosslinked species as indicated in the figure is according to Lupinkova et al. (2004) “Oxidative modifications of the Photosystem II D1 protein by reactive oxygen species: from isolated protein to cyanobacterial cells”, Photochemistry & Photobiology, Vol. 79, pp. 152-62. Similar results were obtained with the other strains.

### DETAILED DESCRIPTION OF THE INVENTION

The present invention relates to an alternative electron transfer pathway that has been introduced into photosystem II by means of mutating one of the components of the PSII complex. The present invention discloses for the first time a specific mutation in the D1 protein of PSII that results in the formation of a novel electron conduit, which enables electrons to flow from PSII to water-soluble electron carrier proteins. The present invention is applicable to plants, algae and certain types of bacteria.

Even though the engineered site is highly conserved among oxygenic photosynthetic organisms, and therefore its modification might be deleterious to the protein function, it was surprisingly found that the new pathway does not alter the ability of PSII to perform natural photosynthetic electron transfer, and the resulting organism maintains photoautotrophic growth. In addition, the variant D1 was found to have increased stability against photodamage in the presence of a water-soluble protein electron carrier.

The natural photosynthetic system has four tunable components that together provide an optimal system for energy conversion: i) Efficient light absorption is performed by pigments bound to antenna light-harvesting complexes (LHC). ii) The LHCs transfer the absorbed energy into a photochemical reaction center that is tuned to quickly perform charge separation. The potential for photochemistry by special chlorophyll molecules present at the reaction center is a result of exquisite positioning of protein residues that tune the electronic state of the donor and nearby intermediate acceptor molecules; iii) The internal electron transfer chain, on both the acceptor and donor sides of the primary donor, are insulated against spurious electron transfer to alternative acceptors. This is achieved by precise positioning of the correct chemical functionalities along the pathway and stabilization of reduced states. Insulation of the pathway is not a simple task, since certain side chains are quite amenable to electron transfer; and iv) The ability to self-assemble the unique surroundings of the photochemical reaction center, which is achieved by protein-protein interactions and also interactions between proteins and lipids in the membrane. The mixture of complimentary protein surfaces (contributing rigidity to the cofactors) and the flexibility imparted by the lipid bilayer, enable both exact positioning of the three previously described components and the dynamic movements required to enable efficient forward electron transfer and inefficient back electron transfer. In photosystem II, the need for proton movement to the secondary acceptor Q_{B}, whether it be through a series of residues or via channels to the outer solvent requires additional structural changes that must occur without disturbing the continued flow of electrons.

One could envision several possible locations for the introduction of an alternative and novel electron transfer pathway, but such pathways would most likely be highly deleterious to normal growth. As disclosed herein, PSII was successfully engineered without the loss of cell viability in general, and without interference to photosynthetic autotrophic growth in particular.

PSII contains the highest biological redox potential. However, the high redox potential has drawbacks, since MI is known to be damaged at all light levels and requires an intricate system of protein replacement. As disclosed herein, the variant D1 according the embodiments of the present invention shows increased stability against photodamage in the presence of water-soluble protein electron carriers, for example, cytochrome c.

On the basis of measurement of oxygen evolution from either isolated photosynthetic membranes or PSII from plants, algae or cyanobacteria, it appears that such systems can support photochemically driven currents on the order of 0.5-2 Coulomb/mg chlorophyll in the presence of exogenous electron acceptors. This current can continue over time periods of minutes to hours, depending on the system, photon flux and acceptor type. However, it is clear that once the membranes are disconnected from cellular support and repair systems, photosynthetic activity will decrease and eventually stop. In order to use biological material for the production of energy, there is a need for methods that will extend the life-time of the electron-transfer chain without disturbing the rate of electron transfer. In association with this condition, one of the important observations presented here is that the presence of cytochrome c as an electron acceptor reduces photodamage to the D1 protein in vitro.

As exemplified herein below, site directed mutants have been engineered which retain photosynthetic viability; the engineered electron transfer conduit enables the reduction of an exogenous electron acceptor that has the potential to cycle electrons to secondary electron acceptors; and the novel electron conduit enables the removal of damaging species from PSII, thus preserving its activity.

### DEFINITIONS

As used herein, the term “photosynthetic organism” refers to an organism capable of performing photosynthesis.

As used herein, the term “oxygenic photosynthetic organism” or “organism performing oxygenic photosynthesis” refers to a photosynthetic organism that uses water as an electron source, and generates oxygen as an end product of photosynthesis.

As used herein, the term “electron carrier” refers to a molecule capable of accepting one (or more than one) electrons from another molecule (the “electron donor”), and then ferry these electrons and donate them to another entity.

The terms “mutant” and “variant” are used interchangeably and as used herein refer to a protein which differs from an unaltered, wild-type protein due to one or more amino acid substitutions introduced into the amino acid sequence.

The terms “wild type” and “unaltered sequence” are used interchangeably and as used herein refer to the naturally occurring DNA/protein sequence.

As used herein, the term “gene” has its meaning as understood in the art. In general, a gene is taken to include gene regulatory sequences (e.g. promoters, enhancers, etc.) and/or intron sequences, in addition to coding sequences (open reading frames).

As used herein, the term “isolated” means 1) separated from at least some of the components with which it is usually associated in nature; 2) prepared or purified by a process that involves the hand of man; and/or 3) not occurring in nature.

The terms “polypeptide,” “peptide” and “protein” are used interchangeably herein to refer to a polymer of amino acid residues.

As used herein, the term “DNA construct” refers to an artificially assembled or isolated nucleic acid molecule which comprises the gene of interest.

As used herein, the term “vector” refers to any recombinant polynucleotide construct that may be used for the purpose of transformation, i.e. the introduction of heterologous DNA into a host cell. One exemplary type of vector is a “plasmid”, which refers to a circular double stranded DNA loop into which additional DNA segments can be ligated. Another exemplary type of vector is a viral vector, wherein additional DNA segments can be ligated into the viral genome. Certain vectors are capable of autonomous replication in a host cell into which they are introduced.

As used herein, a “primer” defines an oligonucleotide which is capable of annealing to (hybridizing with) a target sequence, thereby creating a double stranded region which can serve as an initiation point for DNA synthesis under suitable conditions.

The terms “exogenous” or “heterologous”—when used in reference to DNA sequences or genes, refer to a DNA sequence encoding a protein, polypeptide, or a portion thereof, whose nucleotide sequence is not normally found in the host cell, but is introduced by standard gene transfer techniques. A heterologous gene also includes a gene native to an organism that has been altered in some way (for example, mutated).

As used herein, the terms “transformation” refers to the introduction of foreign DNA into cells. The terms “transformants” or “transformed cells” include the primary transformed cell and cultures derived from that cell regardless to the number of transfers. All progeny may not be precisely identical in DNA content, due to deliberate or inadvertent mutations. Mutant progeny that have the same functionality as screened for in the originally transformed cell are included in the definition of transformants.

The term “plant” is used in it broadest sense. It includes, but is not limited to, any species of woody, herbaceous, perennial or annual plant, mosses and ferns. It also refers to a plurality of plant cells that are largely differentiated into a structure that is present at any stage of a plant's development. Such structures include, but are not limited to, a fruit, shoot, stem, leaf, flower petal, etc. The term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue may be in planta, in organ culture, tissue culture, or cell culture.

Variant D1

The present invention discloses variants of the D1 protein having an amino acid other than a basic amino acid at the position indicated as X₄ in the consensus sequence set forth in SEQ ID NO.: 1.

The electrons flow in PSII includes electron transfer from Q_{A} to Q_{B}. The lifetime of Q_{A} is about 100 μsec in the presence of oxidized Q_{B}, while in the presence of Q_{B} site inhibitors, such as 3-(3,4-dichlorophenyl)-1,1-dimethylurea (DCMU) or Atrazine, the lifetime of Q_{A} can be on the order of seconds. Also, thermoluminesence measurements have shown that the redox potential of Q_{A}/Q_{A} is elevated by the presence of the Q_{B} site inhibitor, DCMU, indicating that even small structural changes in the reaction center can induce changes in the electrochemical properties of the cofactors. The long-lived presence of Q_{A}⁻ may increase the possibility of formation of doubly reduced Q_{A} molecule (Q_{A}²⁻), which may be one of the reasons for the phenomenon known as photo-inhibition. Since the lifetime of Q_{A}⁻ can be extensive, it appears that the Q_{A} binding site is efficiently insulated in the natural system, and electrons are not lost to alternative oxidizing pathways in vivo. Exogenous small molecule oxidants such as ferricyanide can, however, overcome the protein insulation and oxidize Q_{A}⁻.

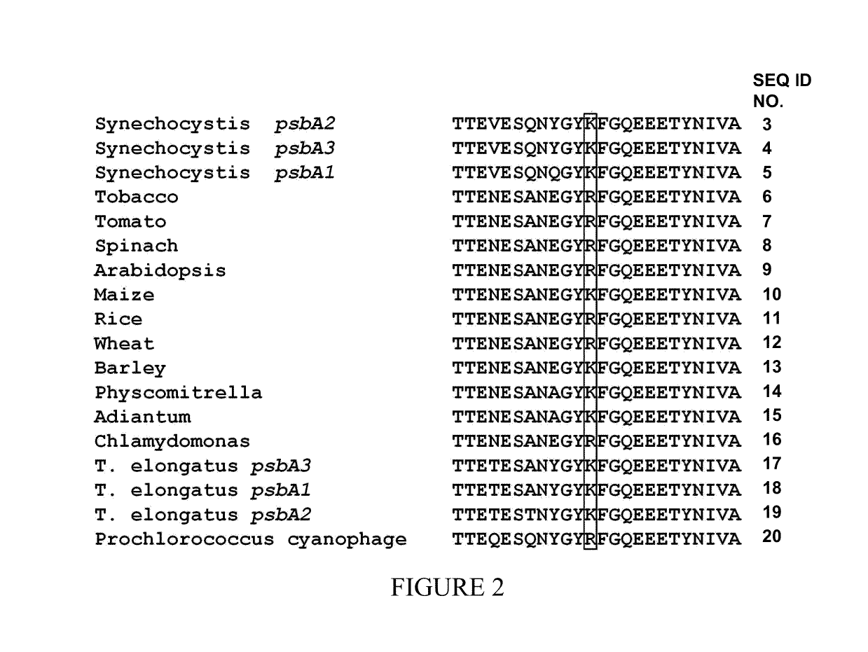

As disclosed herein, a site on the cytoplasmic surface of D1 was identified, that could be modified to result in an alternative electron path. The engineered site is located at the vicinity of the Q_{A} site within PSII. As can be seen from FIG. 2, the site is highly conserved among photosynthetic organisms.

Typically, the D1 protein comprises the following amino acid consensus sequence:

(SEQ ID NO.: 1)
Thr-Thr-Glu-X₁-Glu-Ser-X₂-Asn-X₃-Gly-Tyr-X4-Phe-Gly-Gln-Glu-Glu-Glu-
Thr-Tyr-Asn-Ile-Val-Ala.

wherein:

X₁ represents valine, asparagine, threonine or glutamine;

X₂ represents glutamine, alanine or threonine;

X₃ represents tyrosine, glutamine, glutamate or alanine; and

X₄ represents lysine or arginine.

In principle, X₁ to X₃ may represent any amino acid.

Exemplary amino acid sequences of the conserved area of D1 protein from various oxygenic photosynthetic organisms can be found in FIG. 2. The sequences are set forth as SEQ ID NOs.: 3-20.

The present invention now discloses a variant form of the D1 protein, wherein the above described consensus sequence TTEX₁ESX₂NX₃GYX₄FGQEEETYNIVA comprises a substitution to an amino acid other than a positively-charged amino acid at the position indicated as X₄. X₄ corresponds to position 12 of the consensus sequence. Preferably, the original amino acid present at position X₄ of the consensus sequence is substituted with an acidic amino acid, for example glutamate, aspartate or an equivalent synthetic analogue. In some typical embodiments, the original amino acid present at position X₄ of the consensus sequence is substituted with a glutamate residue or an equivalent synthetic analogue.

Thus, according to a first aspect, the present invention provides an isolated variant D1 protein comprising a mutated SEQ ID NO.: 1 consensus sequence, wherein the amino acid present at the position indicated as X₄ in the consensus sequence set forth in SEQ ID NO.: 1 is substituted with glutamate.

The resulting D1 variant of the present invention comprises the following amino acid sequence:

| | |
|---|---|
| TTEX₁ESX₂NX₃GYEFGQEEETYNIVA. | (SEQ ID NO.: 2) |

wherein:

X₁ represents valine, asparagine, threonine or glutamine;

X₂ represents glutamine, alanine or threonine; and

X₃ represents tyrosine, glutamine, glutamate or alanine.

In principle, X₁ to X₃ may represent any amino acid.

In some embodiments, the consensus sequence corresponds to positions 227-250 of the D1 protein. According to these embodiments, the variant D1 protein comprises a substitution to glutamate at position 238 of D1.

In other embodiments, the consensus sequence corresponds to positions 197-220 of the D1 protein. According to these embodiments, the variant D1 protein comprises a substitution to glutamate at position 208.

In some embodiments, the original, naturally-occurring, amino acid present at the position indicated as X₄ in the consensus sequence set forth in SEQ ID NO.: 1 is lysine. According to these embodiments, an isolated variant D1 protein is provided, the variant comprises a lysine to glutamate substitution at the position indicated as X₄ in the consensus sequence set forth in SEQ ID NO.:1.

In other embodiments, the original, naturally-occurring, amino acid present at the position indicated as X₄ in the consensus sequence set forth in SEQ ID NO.: 1 is arginine. According to these embodiments, an isolated variant D1 protein is provided, the variant comprises an arginine to glutamate substitution at the position indicated as X₄ in the consensus sequence set forth in SEQ ID NO.: 1.

In some embodiments, an isolated variant D1 protein is provided, that contains a lysine to glutamate substitution at position no. 238 of D1 (K238E). In other embodiments, an isolated variant D1 protein is provided, that contains a lysine to glutamate substitution at position no. 208 of D1.

In some embodiments, an isolated variant D1 protein is provided, that contains an arginine to glutamate substitution at position no. 238 of D1 (R238E). In some embodiments, an isolated variant D1 protein is provided, that contains an arginine to glutamate substitution at position no. 208 of D1 (R208E).

In some embodiments, the substitution to glutamate disclosed herein is the only mutation in the variant D1. According to these embodiments, the variant D1 does not contain additional mutations other than the substitution to glutamate disclosed herein.

D1 proteins of all oxygenic photosynthetic organisms may be modified to include the amino acid substitutions disclosed herein. Oxygenic photosynthetic organisms include all photosynthetic organisms that use water as an electron source, and generate oxygen as an end product of photosynthesis, for example, plants, algae and cyanobacteria.

In some embodiments, the D1 protein is selected from a plant, an algal or a cyanobacterial protein.

In some embodiments, the D1 is a plant protein. D1 proteins of both dicotyledons and monocotyledons can be modified according to embodiments of the present invention. In some embodiments, the plants include species of tobacco, tomato, spinach, Arabidopsis, maize, rice, wheat, barley, potato, carrot, cabbage, Physcomitrella and Adiantum. Each possibility represents a separate embodiment of the invention.

In some embodiments, the plant is of a species selected from the group consisting of tobacco, tomato, spinach, Arabidopsis, maize, rice, wheat, barley, potato, carrot, cabbage, Physcomitrella and Adiantum. Each possibility represents a separate embodiment of the invention.

In other embodiments, the D1 protein is an algal protein. In some typical embodiments, the algae belong to the Chlorophyta phylum (chlorophytes). In some embodiments, the alga is of a species selected from Chlamydomonas, Chlorella and Spirogyra. Each possibility represents a separate embodiment of the invention.

In yet other embodiments, the D1 protein is a cyanobacterial protein. In some embodiments, the cyanobacteria include species of Synechocystis, for example Synechocystis sp. PCC 6803, Thermosynechococcus, for example T. elongatus or T. vulcanus and Prochlorococcus. Each possibility represents a separate embodiment of the invention.

In some embodiments, the cyanobacterium is of a species selected from the group consisting of Synechocystis, Thermosynechococcus, and Prochlorococcus. Each possibility represents a separate embodiment of the invention.

An isolated variant D1 may be obtained from a genetically modified organism, for example, those that are described below. Alternatively, an isolated variant D1 may be prepared by any method known to a person skilled in the art, for example, by synthetic methods.

Isolation of the D1 protein may be performed, for example, using strong detergents. Following isolation of photosystem II (as described below), the complex may be dissolved using a strong detergent, including but not limited to, sodium dodecyl sulfate (SDS). The denatured D1 protein can then be isolated. An exemplary method for isolation of the D1 protein using SDS can be found in Adir et al. (1988) “Structural properties of the D1 and surrounding photosystem II polypeptides as revealed by their interaction with cross-linking reagents”, J Biol Chem, Vol. 263 pp. 283-289, the content of which is incorporated by reference herein in its entirety. In this method, the cross-linking agent glutaraldehyde is used to induce cross-linking of all proteins except for the D1 protein, and the D1 protein can then be isolated through gel-electrophoresis.

Photosystem II Complexes

According to another aspect, the present invention provides an isolated PSII comprising the variant D1 of the present invention, as detailed above.

PSII complexes of the present invention enable electron transfer to an exogenous electron carrier. The term “exogenous electron carrier” refers to an electron carrier which is not part of the PSII complex. Typically, the exogenous electron carrier is a water-soluble protein electron carrier. Without wishing to be bound by any particular theory or mechanism, the electron carrier can transiently bind to PSII and abstract the light driven pumped electrons.

In some embodiments, the water-soluble protein electron carrier is selected from the group consisting of cytochrome, ferredoxin, rubredoxin, plastocyanin and flavocytochrome. Each possibility represents a separate embodiment of the invention.

In some embodiments, a cytochrome is used as the electron carrier. In some exemplary embodiments, the cytochrome is selected from the group consisting of cyt c, cyt c₂ and cyt c₆. Each possibility represents a separate embodiment of the invention. In some typical embodiments, cytochrome c is used as the soluble protein electron carrier.

Isolated PSII complexes of the present invention may be obtained from an oxygenic photosynthetic organism that is modified to comprise the variant D1 protein of the present invention. In some embodiments, the PSII complexes are isolated from plants. In other embodiments, the PSII complexes are isolated from algae. In still other embodiments, the PSII complexes are isolated from cyanobacteria.

PSII complexes may be isolated by any method known to those skilled in the art. An illustrative method for the isolation of PSII from cyanobacteria is described in Schatz et al. (1984) “Extraction and characterisation of oxygen-evolving Photosystem II complexes from a thermophilic cyanobacterium Synechococcus sp.”, Photobiochem Photobiophys, Vol. 7, pp. 1-14, the content of which is incorporated by reference herein in its entirety.

An illustrative method for the isolation of PSII from plants is described in Rukhman et al. (2000) “Analysis of the role of detergent mixtures on the crystallization of the reaction center of Photosystem II”, Photosynthesis Research, Vol. 65, pp. 249-259, the content of which is incorporated by reference herein in its entirety. In general, isolation of PSII from plants mainly includes treatment of a crude PSII material with detergents such as, for example, dodecyl-β-D-maltoside followed by several purification steps using ion exchange columns and sucrose density gradients.

Thylakoid Membranes

According to yet another aspect, the present invention provides an isolated thylakoid membrane comprising at least one PSII having the variant D1 protein of the present invention.

The thylakoids are specialized membrane structures in which photosynthesis takes place. In plants and algae, the thylakoids are located inside chloroplasts. In cyanobacteria, the thylakoids are found in the cytoplasm as an intracellular membrane system.

Isolated thylakoid membranes of the present invention may be obtained from an oxygenic photosynthetic organism that is modified to comprise the variant D1 protein of the present invention. In some embodiments, the thylakoid membranes are isolated from plants. In other embodiments, the thylakoid membranes are isolated from algae. In still other embodiments, the thylakoid membranes are isolated from cyanobacteria.

Exemplary methods for thylakoid membranes isolation can be found, for example, in Komenda at al. (2000) “Degradation of the Photosystem II D1 and D2 proteins in different strains of the cyanobacterium Synechocytis PCC 6803 varying with respect to the type and level of psbA transcript”, Plant Molecular Biology 42, 635-45, the content of which is incorporated by reference herein in its entirety.

In general, isolation of thylakoid membranes mainly includes disruption of the cells containing same (for example, by sonication or pressure cells) and separation of the thylakoids by differential centrifugation.

As exemplified herein below, isolated thylakoid membranes of the present invention are able to perform light-dependent reduction of protein electron carrier, for example, cytochrome c, with water as the electron donor. Thus, PSII complexes of the present invention may be utilized in the form of a crude membrane preparation without difficult isolation or manipulation procedures. This is in contrast with synthetic devises that typically require the presence of relatively rare, expensive and potentially polluting components (such as ruthenium, cadmium, telluride, indium, etc.) serving as electron donors and/or acceptors.

Genetically Modified Organisms

The present invention further provides genetically modified oxygenic photosynthetic organisms comprising the variant D1 of the present invention. The gene encoding the wild-type D1 has been mutated in these organisms to encode the variant D1 protein of the present invention.

In cyanobacteria, the gene encoding the D1 protein is located within the genome of the organism. In plants and algae, the gene encoding the D1 protein is typically located within the chloroplast genome.

In some embodiments, more than one copy of the gene encoding the wild-type D1 is present in the genome of the oxygenic photosynthetic organism or its chloroplast genome. According to these embodiments, a genetically-modified oxygenic photosynthetic organism is provided, comprising at least one copy of a mutated D1 gene, the mutated D1 gene encodes a variant D1 protein of the present invention.

Any oxygenic photosynthetic organism may be used according to embodiments of the present invention.

In some embodiments, the genetically modified oxygenic photosynthetic organism is a unicellular organism.

In other embodiments, the genetically modified oxygenic photosynthetic organism is a multicellular organism. According to these embodiments, a genetically modified oxygenic photosynthetic organism is provided, comprising at least one cell comprising at least one copy of a mutated D1 gene, wherein the mutated D1 gene encodes a variant D1 of the present invention. It is to be understood that “at least one cell” refers to a cell capable of performing photosynthesis. For example, if the organism is a plant, cells capable of performing photosynthesis include, but are not limited to, cells of leaves and stems in plants.

In some embodiments, the genetically modified oxygenic photosynthetic organism is a cyanobacterium. The cyanobacterium may be selected from species of Synechocystis, for example Synechocystis sp. PCC 6803, Thermosynechococcus, for example T. elongatus or T. vulcanus and Prochlorococcus. Each possibility represents a separate embodiment of the invention.

In other embodiments, the genetically modified oxygenic photosynthetic organism is an alga. In some typical embodiments, the algae belong to the Chlorophyta phylum (chlorophytes). In some embodiments, the alga is selected from species of Chlamydomonas, Chlorella and Spirogyra. Each possibility represents a separate embodiment of the invention.

In yet other embodiments, the genetically modified oxygenic photosynthetic organism is a plant. In some embodiments, the plant is selected from species of tobacco, tomato, spinach, Arabidopsis, maize, rice, wheat, barley, potato, carrot, cabbage, Physcomitrella, Adiantum. Each possibility represents a separate embodiment of the invention. D1 proteins of both dicotyledons and monocotyledons can be modified according to embodiments of the present invention.

The following description provides exemplary procedures for producing genetically modified oxygenic photosynthetic organism according to the present invention. These exemplary procedures may be modified according to the organism that is used, as known to a person skilled in the art.

A DNA encoding the wild-type D1 protein may be amplified from the genome/chloroplast genome of the selected organism using various methods well known in the art. For example, a DNA encoding the wild-type D1 may be amplified by polymerase chain reaction (PCR) using specific primers, constructed on the basis of the nucleotide sequence of the known wild type sequence. Suitable techniques are well known in the art, described for example in U.S. Pat. Nos. 4,683,195; 4,683,202; 4,800,159 and 4,965,188, the contents of which are incorporated by reference herein in its entirety.

The entire gene encoding the wild-type D1 may be amplified. Alternatively, only part of the gene is amplified, which contains the target site for mutation and flanking regions. An additional alternative is to amplify the gene encoding D1 and upstream and downstream flanking sequences.

Optionally, the genomic DNA/chloroplast DNA is extracted from cells prior to the amplification. See for example, Williams et al. (1988) Methods in Enzymology, vol. 167, pp. 85, the content of which is incorporated by reference herein in its entirety.

The resulting amplified construct may be cloned into a vector and subjected to site-directed mutagenesis. The wild-type codon encoding the amino acid which is present at position X₄ of the consensus sequence set forth in SEQ ID NO.: 1 is replaced by a codon for glutamate. Each of the two known codons for glutamate, namely GAA and GAG, can be used to encode the glutamate residue.

Exemplary methods for site-directed mutagenesis can be found, for example, in Kunkel et al. (1985) Proc. Natl. Acad. Sci. USA, Vol 82, pp. 488-492; Weiner et al. (1994) Gene, Vol. 151 pp. 119-123; and Ishii et al. (1998) Methods Enzymol., Vol. 293, pp. 53-71), the contents of which are incorporated by reference herein in its entirety.

For example, introduction of mutations can be performed using commercially available kits, such as the QuickChange® site-directed mutagenesis kit (Stratagene).

An alternative method to producing a polynucleotide with the desired sequence is the use of a synthetic gene. A polynucleotide encoding a variant D1 protein of the invention may be prepared synthetically, for example using the phosphoroamidite method (see, for example, Beaucage et al., Curr Protoc Nucleic Acid Chem. 2001 May; Chapter 3:Unit 3.3; Caruthers et al. (1987) Methods Enzymol., Vol. 154, pp. 287-313, the contents of which are incorporated by reference herein in its entirety.).

Cloning of the construct encoding the variant D1 protein into a vector can be performed by any known method of constructing a recombinant vector (see for example, Sambrook et al. (1989) Molecular Cloning: A Laboratory Manual, 2d edition, Cold Spring Harbor Press, the content of which is incorporated by reference herein in its entirety). Such methods include, for example, direct cloning, site specific recombination using recombinases and homologous recombination.

The DNA construct may be cloned directly into a vector suitable for transformation. The suitable vector is selected according to the particular gene transfer technique and the organism that is used, as known to a person skilled in the art.

The vector may include a selectable marker. The vector may also include regulatory elements that control its replication in the target cell.

In addition, the DNA construct comprising the gene encoding the variant D1 may also include a selectable marker.

The constructed vector can then be introduced into cells by any means appropriate for the transfer of DNA into cells.

Methods for transforming cyanobacteria are well known in the art. Exemplary procedure is provided below.

Methods for chloroplast transformation can be found, for example in U.S. Pat. Nos. 7,803,991, 4,945,050 and 5,693,507, the contents of which are incorporated by reference herein in its entirety.

Additional example can be found in U.S. Pat. Nos. 5,451,513; 5,545,817; 5,545,818, the contents of which are incorporated by reference herein in its entirety.

Chloroplast transformation may be performed with isolated chloroplasts, which are then integrated into a plant protoplast. Alternatively, chloroplast transformation may be performed with plant tissues.

Introduction of the exogenous DNA into plant cells can be performed by methods of direct DNA transfer. For example, in electroporation, the protoplasts are briefly exposed to a strong electric field, opening up mini-pores to allow DNA to enter. In microinjection, the DNA is mechanically injected directly into the cells using micropipettes. In microparticle bombardment, the DNA is adsorbed on microprojectiles such as magnesium sulfate crystals or tungsten particles, and the microprojectiles are physically accelerated into cells or plant tissues.

Introduction of the exogenous DNA into plant cells can also be performed by Agrobacterium-mediated gene transfer.

The mutated gene may be integrated to the genome/chloroplast genome by homologous recombination. In chloroplast, for example, homologous recombination is readily effected by enzymes inherent to the chloroplast.

The integration of the mutated gene into the genome/chloroplast genome by recombination may be verified by any method known to a person skilled in the art.

Example of such methods include 1) observing shifts in mobility of single-stranded DNA on gel electrophoresis; and 2) amplifying all or part of the gene encoding the D1 protein from the genome/chloroplast genome of the genetically modified organisms to produce an amplified sequence and sequencing the amplified sequence.

In the case of plants, after selecting the desired plant material, whole plants can be regenerated. Means for regeneration vary from species to species of plants, but generally a suspension the protoplasts is first provided. Callus tissue is formed and shoots may be induced from callus and subsequently rooted.

Methods for regeneration, development and cultivation of plants from protoplasts can be found, for example, in Weissbach and Weissbach, In.: Methods for Plant Molecular Biology, (Eds.), 1988 Academic Press, Inc., San Diego, Calif., the content of which is incorporated by reference herein in its entirety. This regeneration and growth process typically includes the steps of selection of transformed cells, culturing those individualized cells through the usual stages of embryonic development through the rooted plantlet stage. The resulting transgenic rooted shoots are thereafter planted in an appropriate plant growth medium such as soil.

Plant regeneration from cultured protoplasts is also described in Evans et al. (1983) Handbook of Plant Cell Cultures, Vol. 1: (MacMillan Publishing Co. New York); and Vasil I. R. (ed.), Cell Culture and Somatic Cell Genetics of Plants, Acad. Press, Orlando, Vol. I (1984), and Vol. III (1986), the contents of which are incorporated by reference herein in its entirety.

There is a variety of methods in the art for the regeneration of plants from plant tissue. The particular method of regeneration will depend on the starting plant tissue and the particular plant species to be regenerated, as known to a person skilled in the art.

Methods and Systems for Electron Transfer

The single modification to the D1 protein disclosed herein enables electrons to flow out of the thylakoid membrane instead of being trapped inside it. Thus, photosystem II complexes comprising the variant D1 protein of the present invention may be implemented in methods and systems for electron transfer.

According to another aspect, the present invention provides a method for electron transfer comprising combining:
i) a PSII having the variant D1 protein of the present invention; and
ii) a water-soluble protein electron carrier,
   thereby generating an electron transfer chain enabling electrons to flow from PSII to the water-soluble protein electron carrier.

In some embodiments, the method further comprises combining an inhibitor of the Q_{B} site within D1. Examples of inhibitors of the Q_{B} site include DCMU, atrazine-types (triazines), bromoxynil-types (nitriles) and substituted 9,10 anthraquinones.

The addition of the Q_{B} site inhibitor may increase the rate of electron transfer to the exogenous electron carrier.

According to a further aspect, the present invention provides a system for electron transfer comprising a PSII comprising a variant D1 protein, the variant D1 comprises a substitution to glutamate at the position indicated as X₄ in the consensus sequence set forth in SEQ ID NO.: 1.

According to a further aspect, the present invention provides a system for electron transfer comprising:

a PSII comprising a variant D1 protein, the variant D1 comprises a substitution to glutamate at the position indicated as X₄ in the consensus sequence set forth in SEQ ID NO.: 1; and

a water-soluble protein electron carrier.

In some embodiments, the system further comprises an inhibitor of the Q_{B} site within D1.

The PSII complexes of the present invention may be utilized in the form of a crude thylakoid membrane preparation. Thus, the above-described systems and methods for electron transfer may comprise thylakoid membranes comprising at least one PSII complex of the present invention.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

Methods

Strain and Culture Conditions:

Synechocystis sp. PCC 6803 (wt), RSS and mutant strains were grown in BG-11 medium under white light (50 μE/(m²·sec)) at 27° C. The triple mutant, strain TD34 in which each one of the three psbA genes was replaced with a gene conferring resistance to antibiotics, was grown in BG-11 medium supplemented with 5 mM glucose.

BG-11 media recipe can be found in The SAG Culture Collection (Sammlung von Algenkulturen Göttingen), Culture Collection of Algae, Version 03.2007. 20. BG 11 Medium for Cyanobacteria. Media are usually prepared from stock solutions of macronutrients, trace metals, and vitamins which are added to a large proportion of the final volume of water in order to avoid precipitation. Media may be used as liquid or solidified by 1.0-1.5% agar.

| | Stock solution | Nutrient solution |
|---|---|---|
| | (g/100 ml) | (ml) |
| NaNO₃ | 15 | 10 |
| K₂HPO₄•3H₂O | 0.4 | 10 |
| MgSO₄•7H₂O | 0.75 | 10 |
| CaCl₂•2H₂O | 0.36 | 10 |
| Citric acid | 0.06 | 10 |
| Ferric ammonium citrate | 0.06 | 10 |
| EDTA (dinatrium-salt) | 0.01 | 10 |
| Na₂CO₃ | 0.2 | 10 |
| Micronutrient solution * | | 1 |
| De-ionized or distilled | 919 | |
| water | | |

* Composition of the micronutrient solution (from Kuhl and Lorenzen (1964) Handling and culturing of Chlorella. In: D. M. Prescott, ed., Methods in cell physiology. Vol. 1, pp. 152-187, Academic Press, New York and London).

Add to 1000 ml of de-ionized or distilled water:

| | | |
|---|---|---|
| H₃BO₃ | 61.0 | mg |
| MnSO₄•H₂O | 169.0 | mg |
| ZnSO₄•7H₂O | 287.0 | mg |
| CuSO₄•5H₂O | 2.5 | mg |
| (NH₄)₆Mo₇O₂₄•4H₂O | 12.5 | mg |

Cloning and Site Directed Mutagenesis:

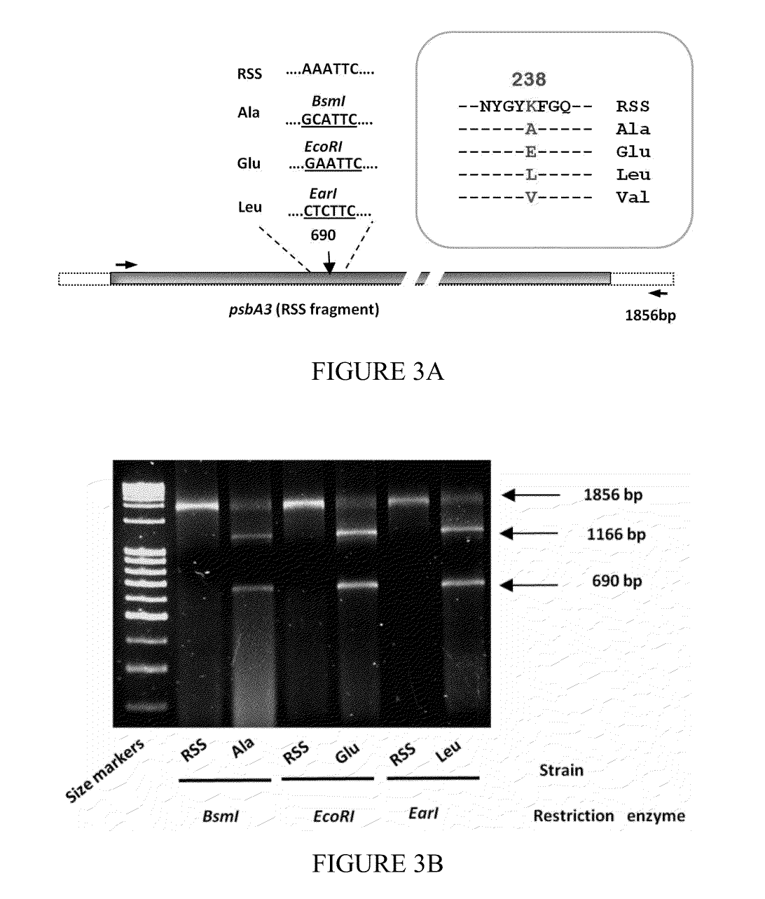

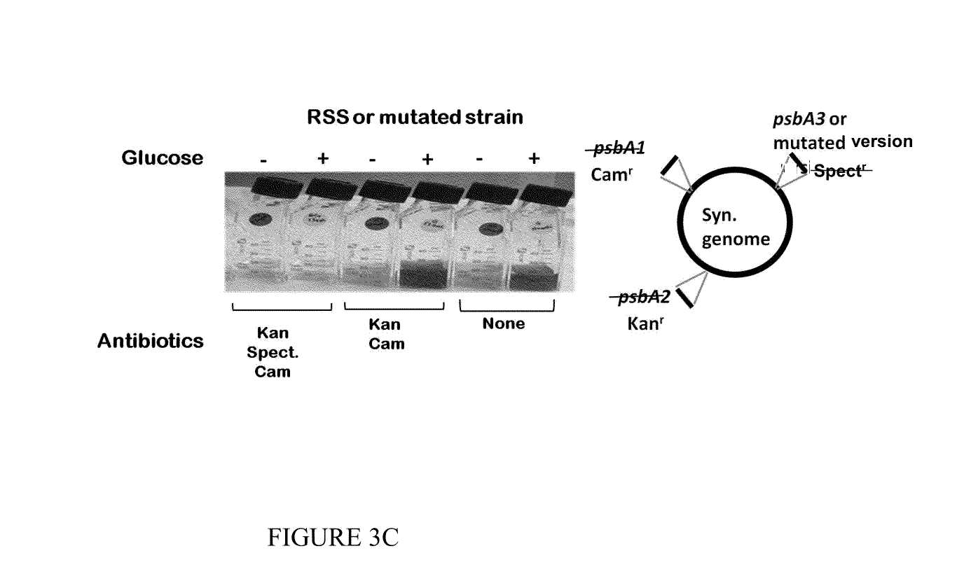

Genomic DNA was isolated from Synechocystis as described in Williams et al. (1988) Methods in Enzymology, vol. 167, pp. 85. A 1795 fragment containing the psbA3 gene (the gene encoding D1) surrounded by 388 bp and 324 bp up and downstream respectively, termed the RSS fragment, was amplified from wild type genome by PCR using the oligonucleotieds shown as “RSS fragment—Forward primer” and “RSS fragment—Reverse primer” in Table 1 below. The sequence of the amplified fragment is provided below (SEQ ID NO.: 21). This fragment was cloned into a pSC-A plasmid (Invitrogen) and subjected to site directed mutagenesis by using QuikChange® (Stratagene) and the oligonucleotides displayed in Table 1: the wild type codon AAA coding for lysine was replaced by the codon GCA for Alanine (using “K to A” oligonucleotide), GAA for Glutamic acid (using “K to E” oligonucleotide) or CTC for Leucine (using “K to L” oligonucleotide) (FIG. 3A).

The sequence of the RSS fragment, as cloned into the pSC-A plasmid (SEQ ID NO.: 21):

5′-
GCTTGGCTGGACTGACACTAAAACTCGGTGACTTTTCATCGTGGAAAGGACATAGGCCAA
GGTGATCCCGCCCCCTTTTCTTGAGCACGACATAATCGCCGATAATTTCGACAATGTCTAT
TCTTTGCTTAATTTCTTGGATGGTGTCTGGATGCAGACGGAGGTTATCCATGATAAATCAA
CCTACTCTCCTTCTAAGAGTGACGATGGGGAGAGATACTTGTCAACTTAAGCTCTACTCAA
GCATGGTCAACCAGCAAAAAGCACCCCCCGGTGAAGGAGAGTGCAATTTGCAGATTATTC
AGTTGGCATTACACCAAGGAATTAACCGTTGACAGCAGGAGCGGTCAAAGCCACAGGAG
CTTGCTCCCCAGACGCTAAGTCGAGGGGGAAGTTGTGGGCATTGCGTTCGTGCATTACTTC
AAAACCGATGTTGGCGCGGTTCAATACATCAGCCCAGGTGCCGATTACACGGCCTTGGCT
ATCCAAGATGGACTGGTTGAAGTTGAAACCGTTCAGGTTGAACGCCATGGTGCTTACACC
CATAGCAGTGAACCAGATGCCGATTACAGGCCAAGCACCCAAGAAGAAGTGCAAGGAAC
GGCTGTTGTTGAAAGAAGCATATTGGAAGATCAACCGACCAAAGTAGCCGTGGGCGGCAA
CGATGTTGTAGGTTTCTTCTTCTTGACCGAATTTGTAACCGTAGTTCTGGGATTCAACTTCG
GTGGTTTCACGCACCAAGGAGGAGGTTACCAAGGAACCGTGCATGGCGGAGAACAAGCT
ACCACCGAATACACCAGCCACACCTAACATGTGGAAGGGGTGCATCAGGATGTTGTGCTC
AGCTTGGAACACGATCATGAAGTTGAAGGTACCAGAAATACCCAAGGGCATACCATCAGA
GAAGGAGCCTTGACCAATGGGGTAGATCAAGAATACAGCGGTGGCAGCGGATACGGGGG
CAGAGTAAGCCACACAAATCCAAGGACGCATACCTAAGCGGTAGGAAAGTTCCCACTGAC
GACCCATGTAGCAGAAAATGCCGATGAGGAAGTGGAATACTACCAACTGGTAAGGACCA
CCGTTGTACAACCACTCATCTAAGGAAGCGGCTTCCCAGATGGGGTAGAAGTGCAAACCG
ATAGCGTTGGAAGAAGGTACAACAGCACCAGAGATGATGTTGTTACCGTAAAGCAAAGA
ACCAGCAACGGGCTCACGGATACCGTCGATGTCAACGGGGGGAGCGGCGATGAAGGCAA
TGATGAAGCAAGTGGTGGCAGTTAAGAGGGTGGGGATCATCAAGGTACCGAACCAACCG
ACATAAATCCGGTTGTTGGTAGAGGTCACCCACTGACAAAACTGTTCCCACAATGAAGCG
CTTTCGCGCTGTTGGAGAGTCGTTGTCATTTGGTTATAATTCCTTATGTATTTGTCAATGTT
CAAAGGATTTGGCCTCAAGCTCAGAGATTTCTTTGCTTGGGAGCCGGTTATGTATATGAGT
AATATAACAAGATTGTAACAATTTGTAAATAGCTTGCTGAAAAAAATCTGTCTAGCCGGT
GGTTGCTAGGGGTTGCCGATCGCCATTAGAGGGGTGATTGATTCCGTTGGGGCTTCAGTCT
GTGGTGGTGGGGAGATGGAAAGCCACTAATCCTGCAACACAACTTCCCCTTGGCGCAGAA
TTTGGAAGATTTCCTTTGCTCCGTCCCATTGGGCAACGGTGGACGGTAGACCTTTGCCAAT
TGGCCCCTGTTGCTCCAGGCTGAGGCAATCTAGACAATGAACGGCAGGGAAA

TABLE 1
Oligonucleotides

| | | SEQ |
|---|---|---|
| | | ID |
| Name | Sequence | NO. |
| RSS fragment - | 5′-GCTTGGCTGGACTGACACTA | 22 |
| Forward primer | | |
| RSS fragment - | 5′-TTTCCCTGCCGTTCATTG | 23 |
| Reverse primer | | |
| K to A | 5′-CCCAGAACTACGGTTACGCATTCGGTCAAGAAGAAGAAACC | 24 |
| K to E | 5′-CCCAGAACTACGGTTACGAATTCGGTCAAGAAGAAGAAACC | 25 |
| K to L | 5′-CCCAGAACTACGGTTACCTCTTCGGTCAAGAAGAAGAAACC | 26 |

Transformation into TD34:

The plasmids carrying the mutant psbA were transformed into the TD34 strain using a standard procedure as described in Pakrasi et al. (1988) EMBO Journal vol. 7, pp. 325-32. Five (5) ml of exponentially grown culture were centrifuged at room temperature (RT) at 6,000 rpm for 15 min. The cells were suspended in fresh BG-11 medium to a density of 10⁹ cells, and exogenous DNA was added to a final concentration of 10 μg/ml. The medium was incubated for 5 hours in light and then spread on BG-11 plates/liquid with antibiotics.

Thylakoid Membrane Isolation and Protein Blotting:

Thylakoid membranes preparation was performed as described in Komenda et al. (2000) Plant Molecular Biology, vol. 42, pp. 635-45.

The procedure included the following steps:
Spin down of cells (250 μg chl)
Washing in a buffer containing 20 mM Tris pH 8, 400 mM NaCl, 5 mM MgCl₂
Resuspention in 0.4 ml of the same buffer.
Addition of the same volume of glass beads
Vortex twice for 90 s with 1 min interruption for cooling on ice
Washing the glass beads with the buffer (until clean)
Centrifugation at 9000 g for 30 s (to remove unbroken cells and debris)
Centrifugation of the supernatant at 15000 g for 15 min
Resuspention of the pellet in 50 mM Tris/HCl, pH 7.5, containing 1 M sucrose

The isolated thylakoids were analyzed by 12% SDS-PAGE containing 6M urea. Proteins were then transferred to a nitrocellulose membrane, which was decorated with antisera against the PSII-D1, PSII-D2, the cytochrome b₆/f-Rieske Fe—S protein (Agrisera), the ATP synthase 13 subunit or the PSI-I protein. For the analysis of the total amount of D1 in the membrane, including the crosslinked species, membranes (0.1 μg chl) were applied to nitrocellulose membrane using a dot-blot apparatus and the amount of D1 detected by specific antibodies (Agrisera).

Photosynthetic Activity Measurements

2,6-dichlorophenolindophenol (DCPIP) Reduction Rates:

5 μg chl of isolated thylakoids from wt or mutant strains were suspended in 50 mM MES buffer (2-(N-morpholino)ethanesulfonic acid) PH=6, 5 mM MgCl₂ and 15 mM NaCl and were incubated with 50 mM DCPIP at 30° C. for 30 sec. in the dark. The absorption of the solution was measured at 598 nm, prior to and following illumination with white light (1000 μE/(m²·sec)) for 50 sec.

Oxygen evolution rates: 250 ml of cells (O.D₇₃₀>1) were harvested, centrifuged at 8000 rpm for 10 min, and resuspended in 30 ml of fresh BG-11 media. The suspension was bubbled with N₂ and then incubated for 5 min at 28° C. in the dark. Rates of oxygen evolution were determined using a Clark-type electrode (Hansatech). Cells were then illuminated with white light for 50 sec and the increase in oxygen concentration was measured. The rate of oxygen evolution was calibrated with solutions containing 0 and 100% saturated oxygen.

Cytochrome c Reduction:

5 μg chl of isolated thylakoids were incubated with 30 μM cytochrome c in a buffer containing 50 mM MES (pH 6), 5 mM MgCl₂ and 15 Mm NaCl, and illuminated for 3 and 5 min with white light. Cytochrome c extinction coefficients were determined by comparison of the reduced minus oxidized absorption spectrum.

Analyzing the Rate of D1 Photo Damage in Illuminated Thylakoids:

Thylakoid membranes (30 μg chl/ml) were illuminated with light intensity of 850 μE/(m²·sec) at 30° C. in the absence or presence of horse heart cytochrome c (0.17 mM) (Sigma). Samples were analyzed by western blot as described above.

Molecular Modeling:

Calculations of all electrostatic potential surfaces were performed using the APBS (Adaptive Poisson-Boltzmann Solver) software package. The coordinates of the 3BZ1-1 PSII structure were modified using PDB2PQR to include hydrogen and partial charges, followed by surface calculation and visualization using the APBS plugin in Pymol. All electrostatic potentials are presented on the same scale. Virtual mutagenesis of D1-238 was performed using Pymol, and the mutated side-chains were positioned in the same orientation as that of D1-Lys238.

### Example 1

### In Silico Design of the Electron Conduit

PSII performs linear electron transfer from the oxygen evolving complex (OEC) to the secondary acceptor, Q_{B}. The redox active components from Y_{z} to Q_{B} are embedded within the D1 and D2 proteins, while the OEC is bound to the luminal face of PSII. The bacterial reaction center of Rhodobacter (Rb). sphaeroides (bRC) shares many physical and functional similarities with PSII. However, unlike PSII, the bRC serves as a component of a cyclic electron transfer system that contains a conduit for electron transfer donation from soluble cytochrome c₂ (cc2) to the oxidized donor, P₈₆₀⁺. The binding of cc2 to the bRC has been studied in the past and the binding site has been determined by X-ray crystallography. The cc2 binding site has a significant negative electrostatic potential which is complementary to the positive electrostatic potential of the cc2 surface. The binding affinity of cc2 to the bRC has been estimated to be on the order of 0.1-1 μM and there is an excess of cc2 in Rb. sphaeroides cells which, together, assure a high turnover rate.

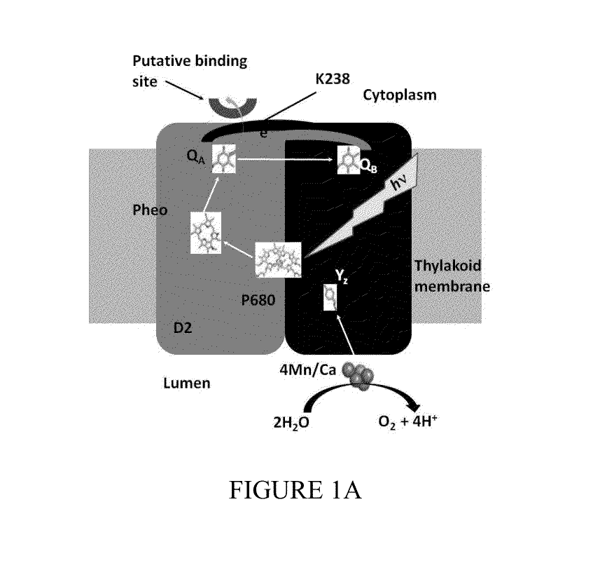

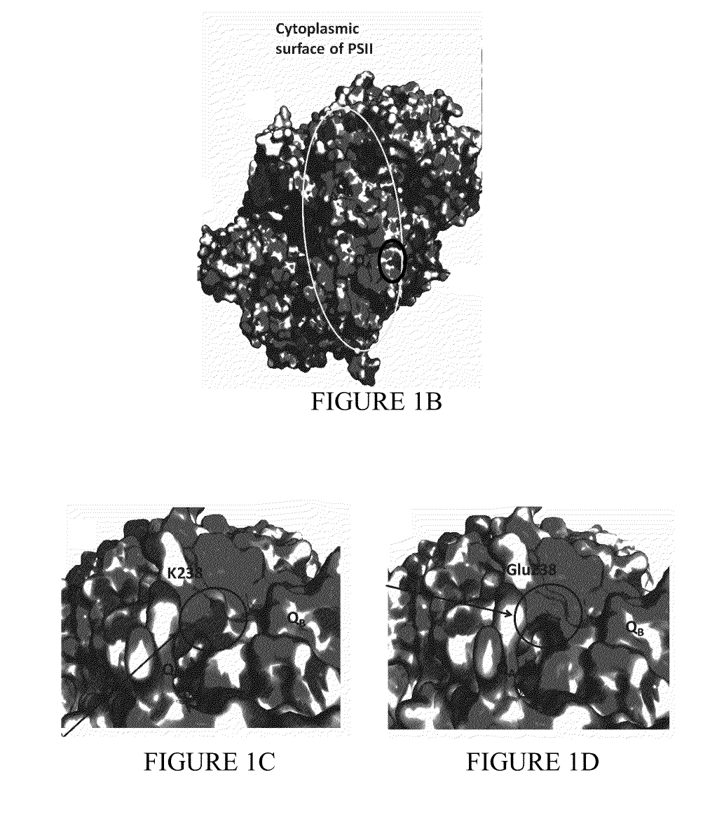

The region of the PSII surface that is homologous to the cc2:bRC binding surface accommodates the oxygen evolving center (OEC) (FIG. 1A). However, the PSII surface that faces the cytoplasm is rather flat and could potentially interact with redox active soluble proteins. In cyanobacteria, this surface is at least partially occupied by the phycobilisome antenna while in green algae and plants, it participates in the formation of the grana stacks. Thus, spurious binding of redox proteins present in the cytoplasm, in vivo is limited. However, isolated thylakoid membranes could present the cytoplasmic surface to electron transfer proteins, essentially “short-circuiting” the natural flow of electrons. The cytoplasmic surface of PSII was examined using the available crystal structures. FIG. 1B shows the calculated electrostatic potential of the PSII surface facing the cytoplasm. From this vantage point, the Q_{A} acceptor site (black single oval) is more negative than the Q_{B} site. A patch of positive potential, located above Q_{A} (FIG. 1C), is due to the presence of D1-Lys238. Interestingly, this position is either a lysine or an arginine in many oxygenic photosynthetic organisms, which would suggest functional importance (FIG. 2). Modifications to this site could be deleterious to PSII activity; however, since D1-Lys238 is located on the surface closest to Q_{A}, an engineered binding site could bring an electron acceptor to within 15-20 Å of Q_{A}. At this distance, the kinetics of electron transfer from Q_{A} would most likely be within the μsec range. The D1-Lys238 residue was virtually mutagenized to either neutral or acidic residues, with different molecular volumes. FIG. 1D shows that the positive patch in the calculated electrostatic potential of the virtual mutant D1-K238E (Glu) was abolished. Neutral mutations (K238A or K238L) were less negative than the Glu mutant (not shown).

### Example 2

### Engineering and Characterization of the Electron Conduit

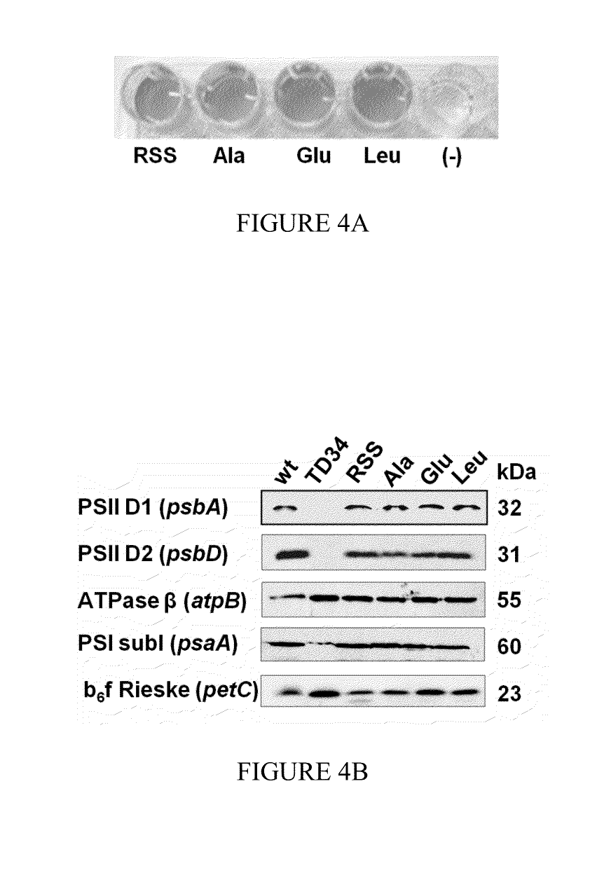

A. Site-specific mutagenesis at the D1-Lys238 site. The cyanobacterium Synechocystis sp. PCC 6803 (Syn), is amenable to site-specific mutagenesis and photoautotrophic/heterotrophic selection procedures. It contains three copies of the psbA gene encoding the D1 protein. In the TD34 strain, each of the three psbA genes have been replaced by antibiotic resistance cassettes This enables the replacement of a single cassette with a wt or mutated copy of psbA, using heterologous recombination. The resulting mutants can either be grown on glucose or photoautotrophically. Three different mutations were introduced into the psbA3 gene D1-Lys238 site: Ala, Glu and Leu. Each of the mutations was verified by PCR, restriction enzyme cleavage, and DNA sequencing (FIGS. 3A and B). The mutated genes were introduced into the TD34 strain and the resulting transgenic strains were then grown photoautotrophically in the presence of the remaining two antibiotics (FIGS. 3C and 4A). As a control, the unmodified psbA3 gene was introduced into the TD34, creating the RSS strain Immunoblot analysis of thylakoids obtained from the mutated strains disclosed comparable accumulation of the four photosynthetic complexes (FIG. 4B).

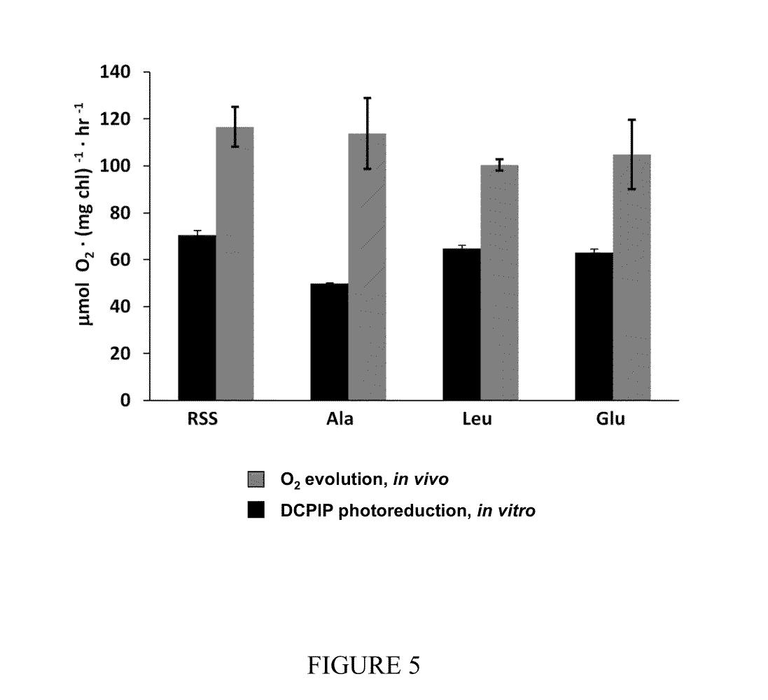

The rates of oxygen evolution of all three viable mutants in vivo FIG. 5, gray bars), and the rates of dichlorophenol indophenol (DCPIP) photoreduction by their isolated membranes in vitro FIG. 5, black bars were approximately 65% of the RSS strain and 50% of the wt Syn rates. Thus, maximal photosynthetic rates were decreased in the mutants; however, the decrease in maximal rate had only a small effect on the rate of cell growth (Table 2 below). Fluorescence induction kinetics of PSII and the thermoluminescence signal attributed to that of the S₂Q_{A}⁻ state were similar in all strains (data not shown).

TABLE 2
Synechocystis sp. PCC 6803 strain growth curves

| | Generation |
|---|---|
| Strain | Strain time (h) * |
| WT | 19 ± 3 |
| RSS | 21 ± 4 |
| Ala | 22 ± 3 |
| Glu | 25 ± 3 |
| Leu | 30 ± 8 |

* average of three experiments

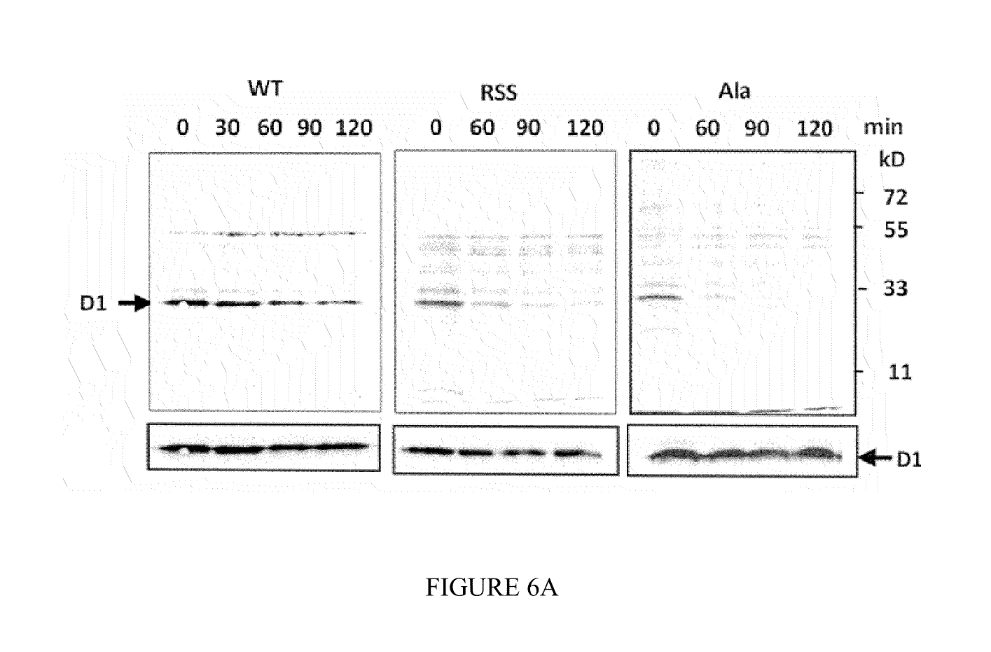

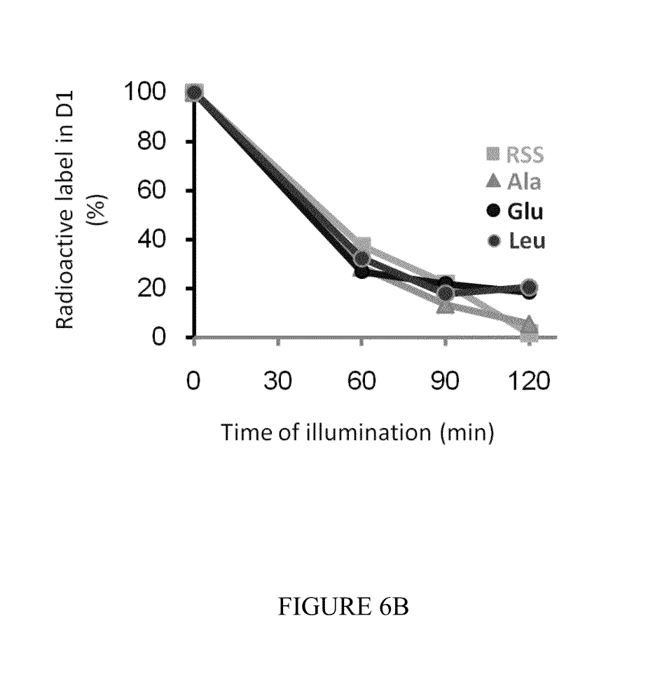

B. During normal PSII activity, the D1 protein is damaged and replaced by an intricate protein synthesis system that avoids the loss of other PSII proteins. Damage to the D1 protein has been suggested to be caused by radicals formed within the RC on account of the elevated redox potential required for electron abstraction from water. In order to see if the site directed lesions introduced into the D1 protein affected the D1 turnover process, Syn strains were labeled with a short pulse of ³⁵S-methionine followed by illumination in the presence of unlabelled methionine. The rate of D1 turnover was quantified by autoradiography and immunoblotting (FIG. 6A). The RSS, Ala, Glu and Leu mutants displayed a similar rate of D1 degradation (FIG. 6B) implying that the degradation machinery is equally capable of processing damaged wt D1 protein and mutated proteins.

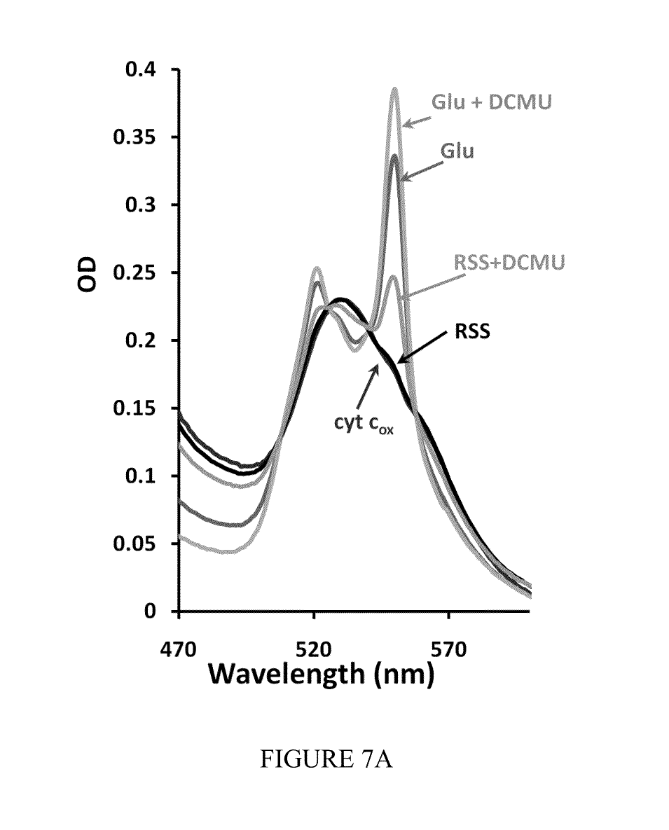

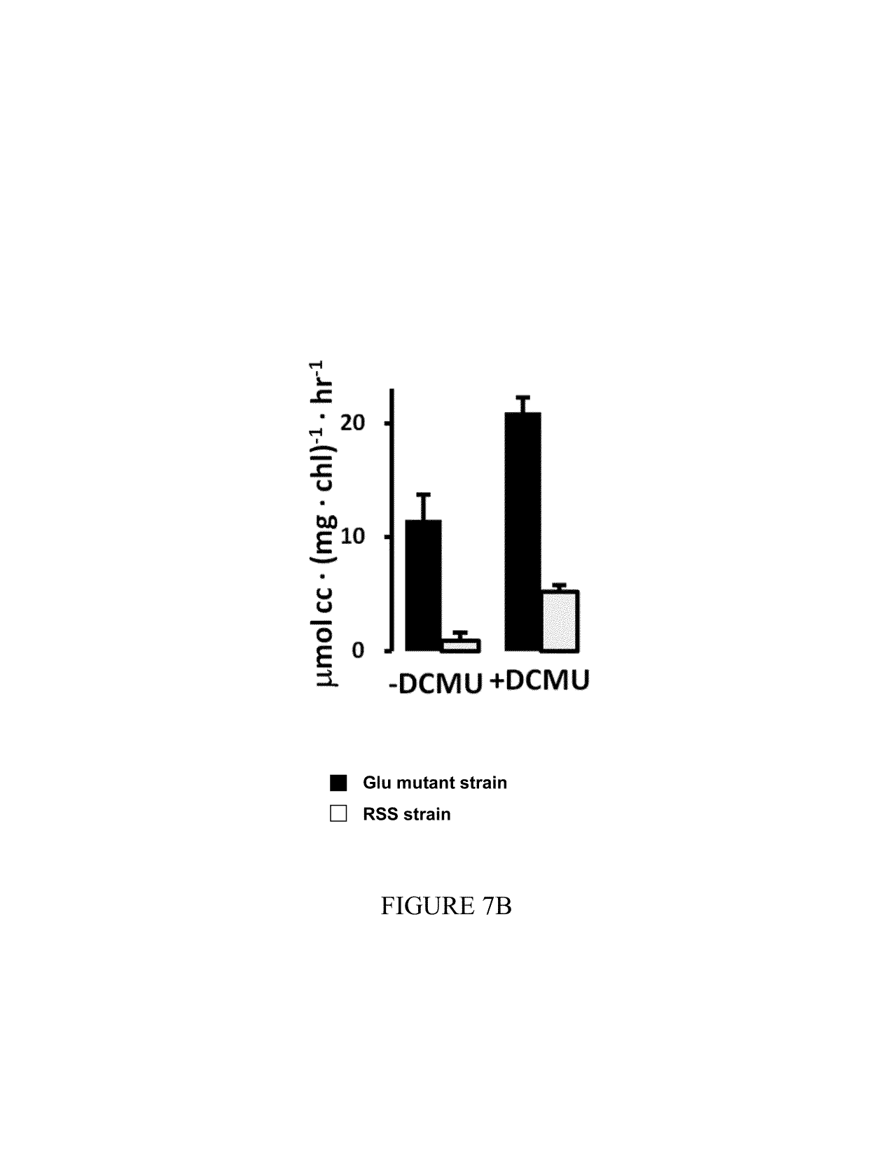

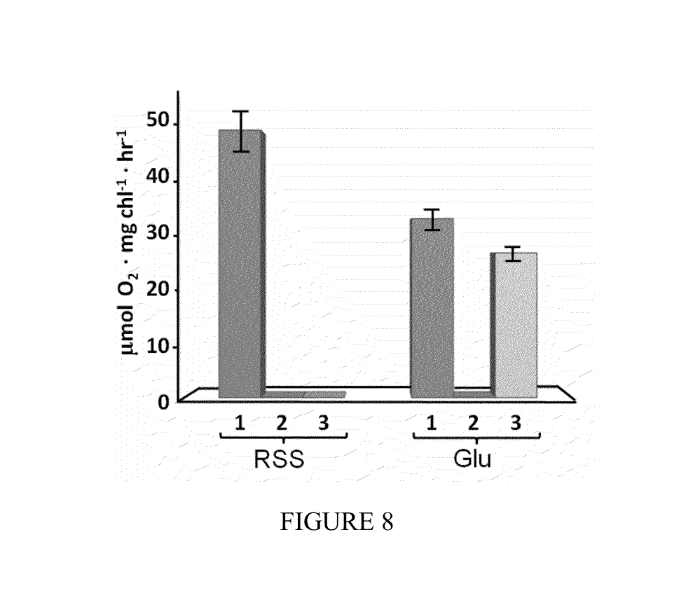

C. Cytochrome c (cc) is an electron shuttle in many bacteria and mitochondria. Oxidized horse-heart mitochondrial cc was incubated with thylakoid membranes isolated from the RSS and mutant strains under standard white light illumination (1000 μE/(m²·sec)). In the measurements of the Glu mutant, a significant increase in the cc a peak at 550 nm was observed, indicating light dependent reduction of cc (FIG. 7A). Addition of DCMU, which blocks electron transfer to Q_{B}, increased the rate of cc reduction from approximately 12 to 20 μmole/(mg chl·hr)(FIG. 7B) Conversely, thylakoids isolated from RSS, Ala and Leu displayed only limited and negligible ability to photoreduce cc, indicating that this phenomenon was acquired by the Glu mutation. The maximal rate of cc reduction reached rates similar to that of oxygen evolution in vitro. These results show that a conduit for electron transfer from Q_{A} to cc have been successfully engineered, while only slightly affecting cell growth. In order to verify that the observed cc reduction was directly related to electrons abstracted from Q_{A} and not another component downstream of Q_{B}, cc photoreduction was measured in the presence of DCMU (FIG. 8). These measurements showed that the source of the reducing electrons was indeed water oxidized by PSII and Q_{A}. Moreover, membranes isolated from the Glu mutant but not from the RSS strain displayed reasonable oxygen evolving activity when supplied with cc as an exogenous electron acceptor in the presence of DCMU (FIG. 8).

### Example 3

### Electron Transfer to Cytochrome c Protects the D1 Protein

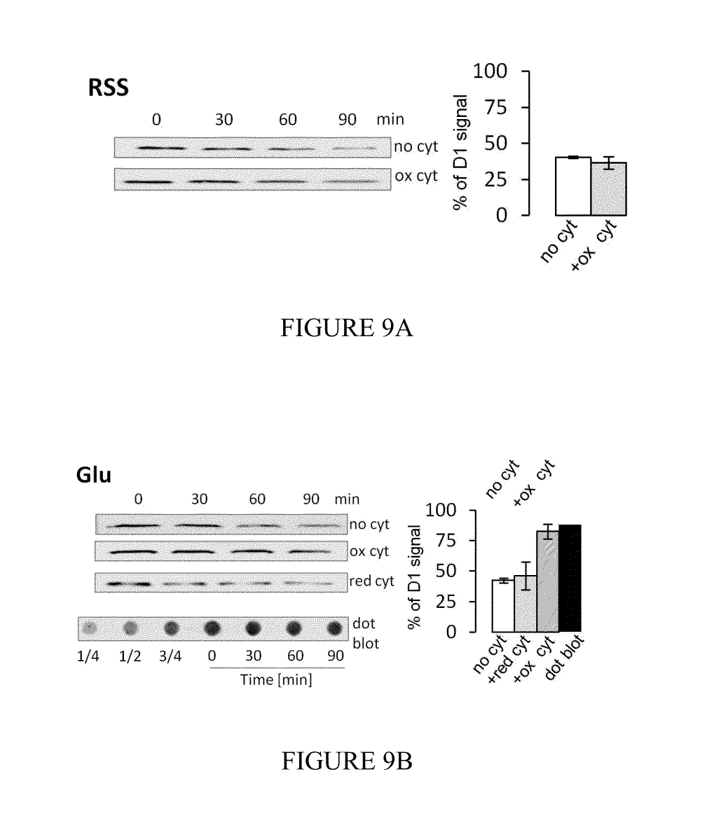

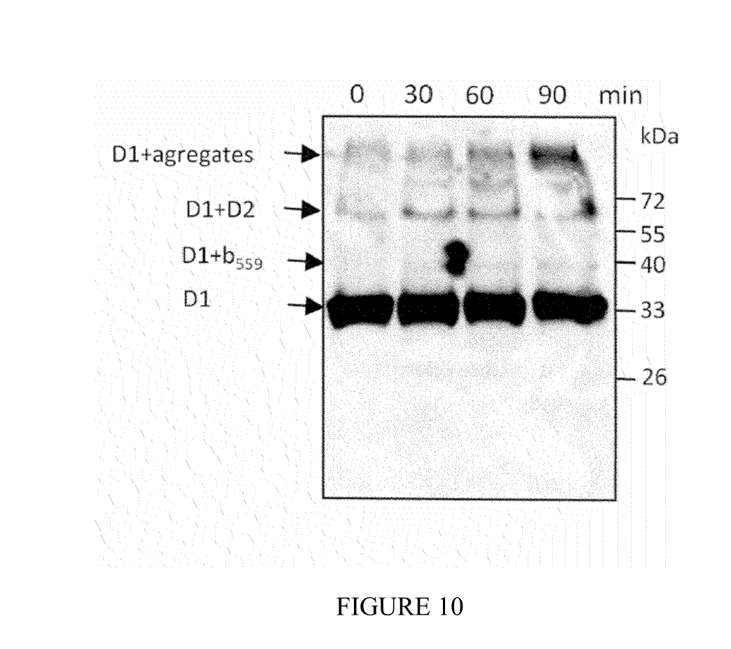

The possibility that the engineered electron transfer conduit could be damaging to PSII was considered. Possible photodamage was initially assessed in the strains by comparing the disappearance rate of the D1 protein 32 kDa band when isolated thylakoids are illuminated. Under such conditions, the degradation of the damaged D1 is significantly inhibited resulting with the crosslinking of the D1 to other PSII proteins, and the disappearance of the corresponding band at 32 kDa. When thylakoids obtained from the RSS or Glu strains were subjected to light, it was found that the 32 kDa D1 band gradually diminished as the D1 was crosslinked at the same rate (FIGS. 9 A and B and FIG. 10). A comparison of the photodamage that occurs when RSS or Glu thylakoid membranes are illuminated either with or without oxidized or reduced cc was then performed. It was found that when incubated with oxidized cc, the D1 protein in thylakoid membranes isolated from the Glu strain (but not the RSS strain) was significantly resistant to the crosslinking effect. In the other conditions, the damaged D1 protein was mostly found in large aggregates with the other PSII proteins, due to crosslinking (FIG. 10). These results indicate that the electron flow from H₂O to cc not only does not cause more damage, rather, it awards a certain level of protection from photodamage.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention.

1. A system for electron transfer comprising at least one photosystem II (PSII) comprising a bio-engineered D1 protein in which a D1 protein consensus sequence is mutated, wherein the mutated D1 protein consensus sequence is SEQ ID NO: 1 with X₄ substituted with glutamate, said PSII enables electron transfer from PSII to an exogenous water-soluble protein electron carrier.

2. The system of claim 1, wherein the at least one PSII is from a cyanobacterium, an alga or a plant.

3. The system of claim 2, wherein the at least one PSII is from a cyanobacterium.

4. The system of claim 3, wherein the cyanobacterium is of a species selected from the group consisting of Synechocystis, Thermosynechococcus, and Prochlorococcus.

5. The system of claim 2, wherein the at least one PSII is from an alga.

6. The system of claim 5, wherein the alga is of a species selected from the group consisting of Chlamydomonas, Chlorella and Spirogyra.

7. The system of claim 2, wherein the at least one PSII is from a plant.

8. The system of claim 7, wherein the plant is of a species selected from the group consisting of tobacco, tomato, spinach, Arabidopsis, maize, rice, wheat, barley, potato, carrot, cabbage, Physcomitrella and Adiantum.

9. The system of claim 1, comprising isolated thylakoid membranes comprising the at least one PSII comprising said bio-engineered D1 protein.

10. The system of claim 1, comprising a genetically modified oxygenic photosynthetic organism comprising at least one copy of a mutated D1 gene, the mutated D1 gene encodes the bio-engineered D1 protein.

11. The system of claim 10, wherein the oxygenic photosynthetic organism is a unicellular organism.

12. The system of claim 10, wherein the oxygenic photosynthetic organism is a multicellular organism.

13. The system of claim 12, wherein the organism comprises at least one cell comprising at least one copy of the mutated D1 gene.

14. The system of claim 1, wherein the D1 protein consensus sequence is located at residues 227-250 of the bio-engineered D1 protein, and the glutamate substitution is at residue 238.

15. The system of claim 1, further comprising a water-soluble protein electron carrier.

16. The system of claim 15, wherein the water-soluble protein electron carrier is selected from the group consisting of cytochrome, ferredoxin, rubredoxin, plastocyanin and flavocytochrome.

17. The system of claim 16, further comprising an inhibitor of the Q_{B} site of D1 protein.

18. A method for electron transfer comprising combining:
i) a photosystem II (PSII) comprising a bio-engineered D1 protein in which a D1 protein consensus sequence is mutated, wherein the mutated D1 protein consensus sequence is SEQ ID NO: 1 with X₄ substituted with glutamate, said PSII enables electron transfer from PSII to an exogenous water-soluble protein electron carrier; and
ii) a water-soluble protein electron carrier,
thereby generating an electron transfer chain enabling electrons to flow from PSII to the water-soluble protein electron carrier.

19. The method of claim 18, further comprising combining an inhibitor of the Q_{B} site of D1 protein.
